(19) United States
(12) Patent Application Publication
(10) Pub. No.: US 2003/0191191 A1
(43) Pub. Date: Oct. 9, 2003

(54) Methods for preventing and treating peripheral neuropathy by administering desmethylselegiline delivery compositions

(76) Inventors: Cheryl D. Blume, Tampa, FL (US); Anthony R. DiSanto, Gobles, MI (US)

Correspondence Address: Margaret J. Sampson, VINSON & ELKINS LLP, 2300 First City Tower, 1001 Fannin, Houston, TX, 77002-6760, US

(21) Appl. No.: 10/382,126

(22) Filed: Mar. 4, 2003

Related U.S. Application Data
(63) Continuation-in-part of application No. 09/800,040, filed on Mar. 5, 2001, now Pat. No. 6,375,979.
(62) Division of application No. 09/448,483, filed on Nov. 24, 1999, now Pat. No. 6,210,706.
(62) Division of application No. 08/679,328, filed on Jul. 12, 1996, now Pat. No. 6,033,682.
(63) Continuation-in-part of application No. PCT/US96/01561, filed on Jan. 11, 1996.
(63) Continuation-in-part of application No. 08/679,330, filed on Jul. 12, 1996, now Pat. No. 6,348,208.
(60) Provisional application No. 60/361,609, filed on Mar. 4, 2002.
(60) Provisional application No. 60/001,979, filed on Jul. 31, 1995.

(51) Int. Cl. A61K 31/137

(52) U.S. Cl. 514/650

(57) ABSTRACT

The present disclosure is directed to methods for alleviating the symptoms associated with peripheral neuropathy by administering R(−)-desmethylselegiline, S(+) desmethylselegiline, or a combination of the two. The neuropathy may be the result of a genetically inherited condition, a systemic disease, or exposure to a toxic agent. The disclosure is also directed to a method for treating patients with cancer by administering a chemotherapeutic agent known to have a toxic affect on peripheral nerves together with R(−)-desmethylselegiline, S(+) desmethylselegiline, or a mixture of the two.

### STATEMENT REGARDING FEDERALLY SPONSORED RESEARCH OR DEVELOPMENT

Not applicable.

REFERENCE TO A “Microfiche Appendix”

Not applicable.

### BACKGROUND OF THE INVENTION

1. Field of the Invention

The present invention relates to methods and pharmaceutical compositions for using the selegiline metabolite R(−)-desmethylselegiline (also referred to simply as “desmethylselegiline” or “R(−)DMS”) alone; its enantiomer ent-desmethylselegiline (also referred to as “S(+) desmethylselegiline” or “S(+)DMS”) alone; or a combination, such as, for example, a racemic mixture, of the two enantiomers. In particular, the present invention provides compositions and methods for using these agents to prevent or treat peripheral neuropathy, particularly for preventing or alleviating the symptoms associated with peripheral neuropathy caused by disease or exposure to a toxic agent, e.g., a chemotherapeutic agent.

2. Description of Related Art

Peripheral neuropathy is associated with a wide variety of causes, including genetically acquired conditions, systemic disease, and exposure to toxic agents. It can manifest itself as a dysfunction of motor, sensory, sensorimotor, or autonomic nerves.

Among the most important toxic agents causing peripheral neuropathy are therapeutic agents, particularly those used for the treatment of neoplastic disease. In certain cases, peripheral neuropathy is a major complication of cancer treatment and is the main factor limiting the dosage of chemotherapeutic agents that can be administered to a patient (Macdonald, Neurologic Clinics 9:955-967 (1991)). This is true for the commonly administered agents cisplatin, paclitaxel, and vincristine (Broun, et al., Am. J. Clin. Oncol. 16:18-21 (1993); Macdonald, Neurologic Clinics 9:955-967 (1991); Casey, et al., Brain 96:69-86 (1973)). The therapeutic efficacy of chemotherapeutics is typically a function of dose; therefore increasing dosage provides increased patient survival (Macdonald, Neurologic Clinics 9:955-967 (1991); Oxols, Seminars in Oncology 16, suppl. 6:22-30 (1989)). The identification of methods for preventing or alleviating dose-limiting peripheral neuropathologic side effects would allow higher, and thus more therapeutically effective doses of these chemotherapeutics to be administered to patients, i.e.,

Beyond the potential for increasing the effectiveness of cancer chemotherapy, the identification of new methods for treating peripheral neuropathy has obvious value in alleviating the suffering of patients with a wide variety of systemic diseases and genetic conditions. In many cases, progressive neuropathy in the peripheral nervous system can be debilitating or fatal.

Presently there are few drugs that are useful for treating peripheral neuropathy. Examples of drugs that have been shown to be useful in treating peripheral neuropathy include prednisone and IVIg to treat chronic inflammatory or immune-mediated polyneuropathies; cyclophosphamide to treat vasculitic neuropathies; famciclovir, tegretol, tricyclic antidepressants, gabapentin, topical Lidocaine, ribavirin, and other immunomodulatory agents used to treat viral infectious neuropathies; and dapsone, clofazamine, rifampin, nifurtimox, and benznidaxole to treat bacterial infectious neuropathies. Ganciclovir and foscarnet may also be used to treat cytomegalovirus multifocal peripheral neuropathies in patients infected with HIV. Selegiline may also be used to alleviate, reduce, or eliminate symptoms associated with peripheral neuropathy, as described in U.S. Pat. No. 6,239,181, incorporated herein by reference. Peripheral neuropathies may result from, for example, a genetically inherited condition, systemic disease, physical injury, or exposure to a toxic or chemotherapeutic agent.

Two distinct monoamine oxidase enzymes are known in the art: monoamine oxidase A (MAO-A) and monoamine oxidase B (MAO-B). The cDNAs encoding these enzymes show different promoter regions and distinct exon portions, indicating they are encoded independently at different gene positions. In addition, analysis of the two proteins has shown differences in their respective amino acid sequences.

The first compound found to selectively inhibit MAO-B was (R)-N-α-dimethyl-N-2-propynylbenzeethanamine, also known as L-(−)-N-α-N-2-propynylphenethylamine, (−)-deprenil, L-(−)-deprenyl, R-(−)-deprenyl, or selegiline. Selegiline has the following structural formula:

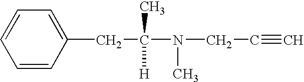

Selegiline is known to be useful when administered to a subject through a wide variety of routes of administration and dosage forms. For example U.S. Pat. No. 4,812,481 (Degussa A G) discloses the use of concomitant selegiline-amantadine in oral, peroral, enteral, pulmonary, rectal, nasal, vaginal, lingual, intravenous, intraarterial, intracardial, intramuscular, intraperitoneal, intracutaneous, and subcutaneous formulations. U.S. Pat. No. 5,192,550 (Alza Corporation) describes a dosage form comprising an outer wall impermeable to selegiline but permeable to external fluids. This dosage form may have applicability for the oral, sublingual or buccal administration of selegiline. Similarly, U.S. Pat. No. 5,387,615 discloses a variety of selegiline compositions, including tablets, pills, capsules, powders, aerosols, suppositories, skin patches, parenterals, and oral liquids, including oil-aqueous suspensions, solutions, and emulsions. Also disclosed are selegiline-containing sustained release (long acting) formulations and devices.

Although a highly potent and selective MAO-B inhibitor, the use of selegiline can be limited by its dose-dependent specificity for MAO-B. The selectivity of selegiline in the inhibition of MAO-B is important to its safety profile following oral administration. Inhibition of MAO-A in peripheral sites (such as, for example, gastric epithelium, liver parenchyma, and sympathetic neurons) may cause toxic side effects by interfering with the metabolism of, for example, dietary tyramine. Tyramine is normally metabolized in the gastrointestinal tract by MAO-A, but when MAO-A is inhibited, tyramine absorption is increased following consumption of tyramine-containing foods such as cheese, beer, herring, etc. This results in the release of catecholamines which can precipitate a hypertensive reaction, referred to as the “cheese effect.” This effect is characterized by Goodman and Gilman as the most serious toxic effect associated with MAO-A inhibitors.

Selegiline is metabolized into its N-desmethyl analog and other metabolites. Structurally, this N-desmethyl metabolite is the R(−) enantiomeric form R(−)DMS of a secondary amine of the formula:

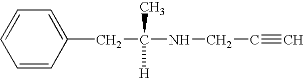

Heretofore, R(−)DMS was not known to have pharmaceutically useful MAO-related effects, i.e., potent and selective inhibitory effects on MAO-B. In the course of determining the usefulness of R(−)DMS for the purposes of the present invention, the MAO-related effects of R(−)DMS were more completely characterized. This characterization has established that desmethylselegiline has exceedingly weak MAO-B inhibitory effects and no advantages in selectivity with respect to MAO-B compared to selegiline.

For example, the present characterization established that selegiline has an IC₅₀ value against MAO-B in human platelets of 5×10⁻⁹ M whereas R(−)DMS has an IC₅₀ value of 4×10⁻⁷ M, indicating the latter is approximately 80 times less potent as an MAO-B inhibitor than the former. Similar characteristics can be seen in the following data measuring inhibition of MAO-B and MAO-A in rat cortex mitochondrial-rich fractions:

TABLE 1
Inhibition of MAO by Selegiline and Desmethylselegiline

| | Percent Inhibition | | | |
|---|---|---|---|---|
| | Selegiline | | R(−)desmethylselegiline | |
| Conc. | MAO-B | MAO-A | MAO-B | MAO-A |
| 0.003 μM | 16.70 | — | 3.40 | — |
| 0.010 μM | 40.20 | — | 7.50 | — |
| 0.030 μM | 64.70 | 0 | 4.60 | — |
| 0.100 μM | 91.80 | — | 6.70 | — |
| 0.300 μM | 94.55 | 9.75 | 26.15 | 0.0 |
| 1.000 μM | 95.65 | 32.55 | 54.73 | 0.70 |
| 3.000 μM | 98.10 | 65.50 | 86.27 | 4.10 |
| 10.000 μM | — | 97.75 | 95.15 | 11.75 |
| 30.000 μM | — | — | 97.05 | — |
| 100.000 μM | — | — | — | 56.10 |

As is apparent from the above table, selegiline is approximately 128 times more potent as an inhibitor of MAO-B relative to MAO-A, whereas R(−)DMS is about 97 times more potent as an inhibitor of MAO-B relative to MAO-A. Accordingly, R(−)DMS appears to have an approximately equal selectivity for MAO-B compared to MAO-A as-selegiline, albeit with a substantially reduced potency.

Analogous results are obtained in rat brain tissue. Selegiline exhibits an IC₅₀, for MAO-B of 0.11×10⁻⁷ M whereas R(−)DMS has an IC₅₀ value of 7.3×10⁻⁷ M, indicating R(−)DMS is approximately 70 times less potent as an MAO-B inhibitor than selegiline. Both compounds exhibit low potency in inhibiting MAO-A in rat brain tissue, 0.18×10⁻⁵ for selegiline, 7.0×10⁻⁵ for R(−)DMS. Thus, in vitro R(−)DMS is approximately 39 times less potent than selegiline in inhibiting MAO-A.

Based on its pharmacological profile as set forth above, R(−)DMS as an MAO-B inhibitor provides no advantages in either potency or selectivity compared to selegiline. Indeed, the above in vitro data suggest that use of R(−)DMS as an MAO-B inhibitor requires on the order of 70 times the amount of selegiline.

The potency of R(−)DMS as an MAO-B inhibitor in vivo has been reported by Heinonen, E. H., et al. (“[R(−)Desmethylselegiline, a metabolite of selegiline, is an irreversible inhibitor of MAO-B in human subjects,” referenced in Academic Dissertation “Selegiline in the Treatment of Parkinson's Disease,” from Research Reports from the Department of Neurology, University of Turku, Turku, Finland, No.33 (1995), pp. 59-61). According to Heinonen, R(−)DMS in vivo has only about one-fifth the MAO-B inhibitory effect of selegiline, i.e., a dose of 10 mg of desmethylselegiline would be required for the same MAO-B effect as 1.8 mg of selegiline. In rats, Borbe reported R(−)DMS to be an irreversible inhibitor of MAO-B, with a potency about 60 fold lower than selegiline in vitro and about 3 fold lower ex vivo (Barbe, H. O., J. Neural Trans. (Suppl.):32:131 (1990)). Thus, all these previous investigators have reported data indicating that R(−)DMS is a less-preferred, less effective MAO inhibitor than selegiline and therefore a less desirable therapeutic compound.

### BRIEF SUMMARY OF THE INVENTION

The present invention is based upon the surprising discovery that R(−)DMS and its enantiomer S(+)DMS, having the following structure:

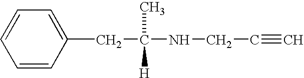

are particularly useful in providing selegiline-like effects in subjects, notwithstanding dramatically reduced MAO-B inhibitory activity and an apparent lack of enhanced selectivity for MAO-B compared to selegiline. Surprisingly, R(−)DMS, S(+)DMS, and combinations such as racemic mixtures of the two are able to alleviate, reduce, or eliminate in whole or in part symptoms associated with peripheral neuropathy. In particular, the disclosure provides a method of protecting a patient from, or treating a patient for, peripheral neuropathy caused by a toxic agent by administering R(−)DMS, S(+)DMS, or a combination of the two in an amount sufficient to prevent, treat, reduce, or eliminate one or more of the symptoms associated with the peripheral neuropathy. Typically, the patient will be a human and the toxic agent will be a chemotherapeutic agent, e.g., an agent administered for the treatment of cancer. Although the method is effective for any toxic chemotherapeutic agent that causes peripheral neuropathy, it is most effective for those agents with particularly severe neuropathic side effects such as cisplatin, paclitaxel, vincristine and vinblastin.

The present disclosure provides novel pharmaceutical compositions in which R(−)DMS, S(+)DMS, or a combination, such as a racemic mixture, of the two is employed as the active ingredient. Also provided are novel therapeutic methods involving the administration of such compositions. More specifically, the present invention provides:

(1) A pharmaceutical composition comprising an amount of R(−)DMS, S(+)DMS, or a combination of the two, such that one or more unit doses of the composition administered on a periodic basis is effective to treat or ameliorate, in whole or in part, peripheral neuropathy in a subject to whom the unit dose or unit doses are administered. This composition may be formulated for non-oral or oral administration.

(2) A method of treating peripheral neuropathy in a subject, such as a mammal, which comprises administering to the mammal R(−)DMS, S(+)DMS, or a combination of the two, in a dosage regimen effective to prevent, treat, reduce, or eliminate, in whole or in part, the peripheral neuropathy, such as a daily dose, administered in a single or multiple dosage regimen of at least about 0.0015 mg, calculated on the basis of the free secondary amine, per kg of the mammal's body weight.

(3) A transdermal delivery system for use in treating peripheral neuropathy in a subject which comprises a layered composite of one or more layers with at least one layer including an amount of R(−)DMS, S(+)DMS, or a combination of the two sufficient to supply a daily transdermal dose of at least about 0.0015 mg of the free secondary amine, per kg of the mammal's body weight.

(4) A therapeutic package for dispensing to, or for use in dispensing to, a subject being treated for peripheral neuropathy. The package contains one or more unit doses, each such unit dose comprising an amount of R(−)DMS, S(+)DMS or a combination of the two, such that periodic administration is effective in treating the subject's peripheral neuropathy. The therapeutic package also comprises a finished a pharmaceutical container containing the unit doses of R(−)DMS, S(+)DMS, or combination thereof, and further containing or comprising labeling directing the use of the package in the treatment of peripheral neuropathy. The unit doses may be adapted for oral administration, e.g. as tablets or capsules, or may be adapted for non-oral administration.

(5) A method of dispensing R(−)DMS, S(+)DMS, or a combination of the two, to a patient being treated for peripheral neuropathy. The method comprises providing patients with a therapeutic package having one or more unit doses of desmethylselegiline, ent-desmethylselegeline or a mixture of the two, in an amount such that periodic administration to the patient is effective in treating peripheral neuropathy. The package also comprises a finished pharmaceutical container containing the desmethylselegiline, ent-desmethylselegeline, or a mixture of the two, and having labeling directing the use of the package in the treatment of peripheral neuropathy. The unit doses in the package may be adapted for either oral or non-oral use.

Preferred embodiments of the present disclosure are methods for preventing or treating peripheral neuropathy caused by a toxic agent; a genetically inherited condition; a systemic disease; or compression, trauma, or entrapment; in a subject in need of such prevention or treatment, by administering to the subject R(−)-desmethylselegiline, S(+)-desmethylselegiline, or a mixture of R(−)-desmethylselegiline and S(+)-desmethylselegiline. Preferably the desmethylselegiline enantiomer or enantiomers are administered in an amount sufficient to prevent, reduce, or eliminate one or more of the symptoms associated with the peripheral neuropathy. In a preferred embodiment, the subject is a mammal, more preferably a human or a domesticated animal.

In a preferred embodiment, the toxic agent that causes peripheral neuropathy is selected from the group consisting of a drug, an industrial chemical, and an environmental toxin. Preferably the drug that causes the peripheral neuropathy that can be treated or prevented by R(−)-desmethylselegiline, S(+)-desmethylselegiline, or a mixture of R(−)-desmethylselegiline and S(+)-desmethylselegiline is chloramphenicol, colchicine, dapsone, disulfiram, amiodarone, gold, isoniazid, misonidazole, nitrofurantoin, perhexiline, propafenone, pyridoxine, phenytoin, simvastatin, tacrolimus, thalidomide, or zalcitabine. In another preferred embodiment, the toxic agent is acrylamide, arsenic, carbon disulfide, hexacarbons, lead, mercury, platinum, an organophosphate, thallium, or a chemotherapeutic agent. Preferably the chemotherapeutic agent is cisplatin, paclitaxel, vincristine, or vinblastin, and the chemotherapeutic agent is being administered for the treatment of cancer in the subject.

In a preferred embodiment, the genetically inherited condition that causes peripheral neuropathy is selected from the group consisting of Charcot-Marie-Tooth Disease, Dejerine-Sottas Disease, Riley-Day Syndrome, Porphyrias, Giant Axonal Neuropathy, and Friedrich's ataxia. In another preferred embodiment, the peripheral neuropathy caused by a systemic disease is selected from the group consisting of acquired primary demyelinating neuropathy, distal symmetric sensory polyneuropathy, distal symmetric sensorimotor polyneuropathy, vasculitic neuropathy, infectious neuropathy, idiopathic neuropathy; immune-mediated neuropathy; nutrition-related neuropathy, and paraneoplastic neuropathy. In a preferred embodiment, the acquired primary demyelinating neuropathy is chronic inflammatory demyelinating polyradiculoneuropathy (CIDP), acute inflammatory demyelinating polyneuropathy (AIDP), or Guillain-Barre syndrome. In another preferred embodiment, the infectious neuropathy is caused by herpes simplex, herpes zoster, hepatitis B, hepatitis C, HIV, cytomegalovirus, diphtheria, leprosy, or Lyme disease. In yet another preferred embodiment, the systemic disease is alcoholic polyneuropathy, diabetes mellitus, uremia, rheumatoid arthritis, sarcoidosis, pernicious anemia, or hypothyroidism. In a preferred embodiment, the compression that causes peripheral neuropathy is selected from the group consisting of carpal tunnel syndrome, ulnar neuropathy at the elbow or wrist, common peroneal nerve at the knee, tibial nerve at the knee, and sciatic nerve.

Another preferred embodiment of the present disclosure is a method for treating a subject with cancer comprising:

a) administering to the subject a chemotherapeutic agent known to have a toxic effect on peripheral nerves, wherein the chemotherapeutic agent is administered at a dose effective at slowing the progression of the cancer; and

b) concurrently administering R(−)-desmethylselegiline, S(+)-desmethylselegiline, or a mixture of R(−)-desmethylselegiline and S(+)-desmethylselegiline to the patient at a dose effective at reducing or eliminating the peripheral neuropathy associated with the chemotherapeutic agent.

If appropriate, the dose of a chemotherapeutic agent may be increased to optimize the therapeutic benefits of the agent while the concurrently administered R(−)-desmethylselegiline, S(+)-desmethylselegiline, or a mixture of R(−)-desmethylselegiline and S(+)-desmethylselegiline functions to minimize the toxic effects of the agent on peripheral nerves. Thus, a higher dose of the chemotherapeutic agent may be administered to a subject while peripheral neuropathy often associated with the higher dose is reduced or eliminated.

Preferred embodiments of the present disclosure are methods for preventing or treating large-fiber peripheral neuropathy, small-fiber peripheral neuropathy, sensory peripheral neuropathy, motor peripheral neuropathy, sensorimotor peripheral neuropathy, or autonomic peripheral neuropathy, in a subject in need of such prevention or treatment, by administering to the subject R(−)-desmethylselegiline, S(+)-desmethylselegiline, or a mixture of R(−)-desmethylselegiline and S(+)-desmethylselegiline. Preferably the desmethylselegiline enantiomer or enantiomers are administered in an amount sufficient to prevent, reduce, or eliminate one or more of the symptoms associated with the particular peripheral neuropathy. In a preferred embodiment, the subject is a mammal, more preferably a human or a domesticated animal.

In a preferred embodiment, the large-fiber peripheral neuropathy is a large-fiber sensory neuropathy or a large-fiber motor neuropathy, that results from abnormal function or pathological change in large, myelinated axons. In another preferred embodiment, the small-fiber peripheral neuropathy results from abnormal function or pathological change in small, myelinated axons, or small, unmyelinated axons. In yet another preferred embodiment, the autonomic peripheral neuropathy results from the dysfunction of peripheral autonomic nerves, and preferably the peripheral autonomic nerves involved are small, myelinated nerves.

Preferred embodiments of the present disclosure are methods for preventing or treating motor neuron disease in a subject in need of such prevention or treatment, by administering to the subject R(−)-desmethylselegiline, S(+)-desmethylselegiline, or a mixture of R(−)-desmethylselegiline and S(+)-desmethylselegiline. Preferably the desmethylselegiline enantiomer or enantiomers are administered in an amount sufficient to prevent, reduce, or eliminate one or more of the symptoms associated with the motor neuron disease. In a preferred embodiment, the subject is a mammal, more preferably a human or a domesticated animal. In another preferred embodiment, the motor neuron disease results from the degeneration of upper motor neurons, lower motor neurons, or upper and lower motor neurons. In yet another preferred embodiment, the motor neuron disease is selected from the group consisting of Progressive Bulbar Palsy, Spinal Muscular Atrophy, Kugelberg-Welander Syndrome, Duchenne's Paralysis, Postpolio Syndrome, Werdnig-Hoffman Disease, Kennedy's Disease, and Benign Focal Amyotrophy.

In preferred embodiments, R(−)-desmethylselegiline or S(+)-desmethylselegiline is administered in a substantially enantiomerically pure form. In other preferred embodiments, R(−)-desmethylselegiline and/or S(+)-desmethylselegiline are administered as the free base or as an acid addition salt. Preferably the acid addition salt is hydrochloride salt. In yet another preferred embodiment, the R(−)-desmethylselegiline, S(+)-desmethylselegiline, or combination of the two is administered orally or non-orally. Preferably, the desmethylselegiline enantiomers are administered by a route that avoids absorption of the desmethylselegiline enantiomers from the gastrointestinal tract. Preferable routs of non-oral administration are transdermal, buccal, sublingual, and parenteral. In yet another preferred embodiment, R(−)-desmethylselegiline and/or S(+)-desmethylselegiline are administered at a dose of between 0.01 mg/kg per day and 0.15 mg/kg per day based upon the weight of the free amine.

Another preferred embodiment of the present disclosure is a pharmaceutical composition that includes R(−)-desmethylselegiline, S(+)-desmethylselegiline, or a mixture of R(−)-desmethylselegiline and S(+)-desmethylselegiline, as well as a second therapeutic agent useful in the treatment of peripheral neuropathy. In a preferred embodiment, one or more therapeutic agents are included in the pharmaceutical composition. In another preferred embodiment, the R(−)-desmethylselegiline, S(+)-desmethylselegiline, or combination of R(−)-desmethylselegiline and S(+)-desmethylselegiline, and the second therapeutic agent, are present in the pharmaceutical composition in an amount such that one or more unit doses of the composition are effective to treat, prevent, reduce, or eliminate peripheral neuropathy in a subject. In other preferred embodiments, R(−)DMS and/or S(+)DMS are administered as the free base or as an acid addition salt. Preferably the acid addition salt is hydrochloride salt. In another preferred embodiment of the present disclosure, the second therapeutic agent useful in the treatment of peripheral neuropathy is selected from the group consisting of prednisone, IVIg, cyclophosphamide, famciclovir, tegretol, tricyclic antidepressants, dapsone, clofazamine, rifampin, nifurtimox, benznidaxole, gabapentin, ganciclovir, foscarnet, cidofovir, acyclovir, topical Lidocaine, and ribavirin.

In other preferred embodiments, the R(−)DMS, S(+)DMS, or combination of the two enantioners in a unit dose of the pharmaceutical composition is between about 0.015 and about 5.0 mg/kg, more preferably between about 0.6 and about 0.8 mg/kg, calculated on the basis of the free secondary amine. In another preferred embodiment, the R(−)DMS, S(+)DMS, or combination of the two enantioners in a unit dose of the pharmaceutical composition is between about 1.0 mg and about 100.0 mg, more preferably between about 5.0 mg and about 10.0 mg. In yet another preferred embodiment, the pharmaceutical composition is for oral administration, for non-oral administration, or for transdermal administration. In a preferred embodiment the pharmaceutical composition is a transdermal patch.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: HPLC Chromatogram of Purified R(−)DMS (Microsorb MV Cyano Column). The purity of a preparation of R(−)DMS was determined by HPLC on a Microsorb MV Cyano column and results are shown in FIG. 1. The column had dimensions of 4.6 mm×15 cm. and was developed at a flow rate of 1.0 ml/min using a mobile phase containing 90% 0.01 M H₃PO₄ (pH 3.5) and 10% acetonitrile. The column was run at a temperature of 40° C. and effluent was monitored at a wavelength of 215 nm. The chromatogram shows one major peak appearing at a time of 6.08 minutes and having 99.5% of the total light-absorbing material eluted from the column. No other peak had greater than 0.24%.

FIG. 2: HPLC Elution Profile of R(−)DMS (Zorbax Mac-Mod C 18 Column). The same preparation that was analyzed in the experiments discussed in FIG. 1 was also analyzed for purity by HPLC on a Zorbax Mac-Mod SB-C18 column (4.6 mm×75 mm). Effluent was monitored at 215 nm and results can be seen in FIG. 2. Greater than 99.6% of the light-absorbing material appeared in the single large peak eluting at a time of between 2 and 3 minutes.

FIG. 3: Mass Spectrum of R(−)DMS. A mass spectrum was obtained for purified R(−)DMS and results are shown in FIG. 3. The spectrum is consistent with a molecule having a molecular weight of 209.72 amu and a molecular formula of C₁₂H₁₅N—HCl.

FIG. 4: Infrared Spectrum. (KBr) of Purified R(−)DMS. Infrared spectroscopy was performed on a preparation of R(−)DMS and results are shown in FIG. 4. The solvent used was CDCl₃.

FIG. 5: NMR Spectrum of Purified R(−)DMS. A preparation of purified R(−)DMS was dissolved in CDCl₃ and ¹H NMR spectroscopy was performed at 300 nm. Results are shown in FIG. 5.

FIG. 6: HPLC Chromatogram of S(+)DMS. The purity of a preparation of S(+)DMS was examined by reverse phase HPLC on a 4.6 min×75 min Zorbax Mac-Mod SB-C18 column. The elution profile, monitored at 215 nm, is shown in FIG. 6. One major peak appears in the profile at a time of about 3 minutes and contains greater than 99% of the total light-absorbing material that eluted from the column.

FIG. 7: Mass Spectrum of Purified S(+)DMS. Mass spectroscopy was performed on the same preparation examined in FIG. 6. The spectrum is shown in FIG. 7 and is consistent with the structure of S(+)DMS.

FIG. 8: Infrared Spectrum (KBr) of Purified S(+)DMS. The preparation of S(+)DMS discussed in connection with FIGS. 6 and 7 was examined by infrared spectroscopy and results are shown in FIG. 8.

FIG. 9: In vivo MAO-B Inhibition in Guinea Pig Hippocampus. Various doses of selegiline, R(−)-desmethylselegiline, and S(+)-desmethylselegiline were administered daily to guinea pigs for a period of 5 days. Animals were then sacrificed and the MAO-B activity in the hippocampus portion of the brain was determined. Results were expressed as a percent inhibition relative to hippocampus MAO-B activity in control animals and are shown in FIG. 9. The plots were used to estimate the ID₅₀ dosage for each agent. The ID₅₀ for selegiline was about 0.008 mg/kg; for R(−)DMS, it was about 0.2 mg/kg; and for S(+)DMS, it was about 0.5 mg/kg.

### DETAILED DESCRIPTION OF THE INVENTION

In the following description, reference will be made to various methodologies well known to those skilled in the art of medicine and pharmacology. Such methodologies are described in standard reference works setting forth the general principles of these disciplines.

The present disclosure is directed to the prevention or treatment of peripheral neuropathy using R(−)DMS, S(+)DMS, or a combination of R(−)DMS and S(+)DMS. Peripheral neuropathy is a common feature of many genetically-inherited and systemic diseases. The nervous system is classified into two parts: the central nervous system (CNS) and the peripheral nervous system (PNS). The CNS is made up of the brain and the spinal cord, while the PNS is composed of all other nerves. The CNS is housed within the dorsal cavity of the body, which is made up of the cranial cavity and houses the brain, as well as the vertebral canal, which houses the spinal cord. As used herein, the term “peripheral neuropathy” refers to abnormal function or pathological changes in peripheral nerves. Peripheral nerves that are located in the PNS include but are not limited to the cranial nerves (with the exception of the second), the spinal nerve roots, the dorsal root ganglia, the peripheral nerve trunks and their terminal branches, and the peripheral autonomic nervous system. The CNS uses the peripheral nervous system to communicate with the body. Any damage to the peripheral nervous system impairs this communication.

Peripheral neuropathy, also known as peripheral neuritis, is a manifestation of many disorders that can cause damage to peripheral nerves. Many different symptoms are associated with peripheral neuropathy as the manifestations of this damage. Symptoms vary widely depending upon the cause of the peripheral neuropathy and the particular types of nerves affected. For example, the symptoms may depend on whether the disorder affects sensory nerve fibers, which are the fibers that transmit sensory information from the affected area to the CNS, or motor nerve fibers, which are the fibers that transmit impulses and coordinate motor activity from the CNS to a muscle, or both. Clinical diagnosis of peripheral neuropathy is based on the clinical history of the subject, a physical examination, the use of electromyography (EMG) and nerve conduction studies (NCS), autonomic testing, cerebrospinal fluid analysis, and nerve biopsies. Because so many different disorders manifest themselves as peripheral neuropathy by affecting a range of nerve types, clinical evaluations and diagnosis of the cause of peripheral neuropathy can be challenging.

Peripheral neuropathies can be categorized by the fiber type that is primarily involved. Peripheral nerves are composed of different types of axons. For example, large-fiber peripheral neuropathies typically involve large myelinated axons, including motor axons and sensory axons, that are responsible for carrying the sense of vibration, proprioception, and light touch. Somatic sensory nerves are myelinated fibers with cell bodies in the dorsal root ganglia (dorsal horn). Somatic motor nerve fibers are myelinated with cell bodies in the ventral horn of the spinal cord and brainstem. Small-fiber peripheral neuropathies primarily include the following fiber types: 1) small myelinated axons that include autonomic fibers and sensory axons, and are responsible for carrying the sense of light touch, pain, and temperature; and 2) small, unmyelinated axons that are sensory and subserve pain and temperature sensations. Many visceral nerves are unmyelinated fibers that include a sensory component and a motor component. The dysfunction of any type of peripheral nerves, for example sensory, motor, sensorimotor, autonomic, or enteric, may manifest itself in any of the various symptoms discussed herein.

Peripheral neuropathies include, but are not limited to, hereditary peripheral neuropathies; idiopathic peripheral neuropathies; immune-mediated peripheral neuropathies; infectious peripheral neuropathies; paraneoplastic peripheral neuropathies; toxic, nutritional, and drug-induced peripheral neuropathies; and traumatic and compressive peripheral neuropathies. The objective of the present disclosure is to administer R(−)DMS, S(+)DMS, or a racemic mixture of R(−)DMS and S(+)DMS to prevent, treat, reduce, or eliminate the symptoms associated with peripheral neuropathy.

There are a limited number of ways that nerves in the PNS can respond to injury or damage. In the periphery, cell bodies are typically found in clusters, which are known as ganglia. A nerve is a bundle of axons that travel together in the periphery. An axon is the single process of a nerve cell that under normal conditions conducts efferent (outgoing) nervous impulses away from the cell body, as well as its remaining processes (dendrites), towards target cells. An axon is capable of transmitting a nerve impulse (action potential) over some distance. The efferent nerves control voluntary and involuntary movement. The afferent division of the PNS sends sensory information from the body to the CNS, while the efferent division of the PNS sends information from the CNS to the body. In the PNS, myelinated axons are surrounded by a myelin sheath, which is provided by cells know as Schwann cells. Myelinated axons are wrapped by concentric layers of cell membrane derived from peripheral nervous system Schwann cells. The presence of a myelin sheath around an axon increases the velocity at which it can conduct a nerve impulse down its length. Along the axon, an open space of uninsulated axon occurs between myelin wrappings. Conduction of the nerve impulse increases because the nerve impulse effectively jumps from one space to another between insulating cells.

Axonopathy is damage that occurs at the level of the axons. This damage can result in a disruption of the axon (e.g., by trauma), which can result in degeneration of the axon and the myelin sheath distal to the site of the injury, also called Wallerian degeneration. In many toxic and metabolic injuries to the PNS, the most distal portion of the axons will degenerate, which also results in the breakdown of the myelin sheath (also known as “dying back,” or length-dependent neuropathy). There are also many peripheral neuropathies that involve a mixture of both axonal degeneration and demyelination. Myclinopathies, or acquired demyelinating neuropathies, result in the degeneration of the myelin sheath, while leaving axons relatively untouched. R(−)DMS, S(+)DMS, or a combination of R(−)DMS and S(+)DMS may also be able to treat peripheral neuropathy by increasing the survival of Schwann cells, thereby decreasing the demyelination of axons. Neuronopathies occur at the level of dorsal root ganglia or motor neuron, with a subsequent degeneration of peripheral processes.

Peripheral neuropathy may involve damage to a single nerve or nerve group (mononeuropathy), or it may involve multiple nerves (polyneuropathy). Peripheral neuropathies may be focal, multifocal, symmetric, or non-symmetric, and can be cause by a pressure injury, for example by a direct injury or compression of the nerve by other nearby body structures. Trauma, compression, and entrapment are common causes of focal nerve injuries. Compression can be caused by peripheral nerve tumors, tumors that press on nerve tissue, abnormal bone growth, cysts or other collections of fluid or tissue that press on nerves, casts, splints, braces, crutches, or other appliances. Nerve injury can also occur from being in a cramped position or in one position for a prolonged periods of time. Entrapment peripheral neuropathy may occur from compression of a nerve when it passes through a narrow space, and mechanical factors may be complicated by ischemia.

One category of peripheral neuropathies are focal neuropathies. Focal peripheral neuropathies include but are not limited to common compression neuropathies, and may involve acute arterial occlusion, carpal tunnel syndrome, ulnar neuropathy at the elbow (tardy unlar palsy) or wrist, proximal median nerve at the elbow, median nerve at the wrist, anterior interosseous nerve, radial nerve in the upper arm, sciatic nerve, peroneal neuropathy at the fibular head or knee, tibial nerve at the knee, lateral femoral cutaneous nerve (meralgia paresthetica), lateral cutaneous nerve at the thigh, or spinal accessory nerve in posterior cervical triangle of the neck. Additionally, ischemia is thought to be the basis of the mild distal peripheral neuropathy of polycythemia.

Another class of peripheral neuropathies are sensory neuropathies. Sensory neuropathy typically involves a dysfunction or damage of peripheral sensory neurons, which may manifest as a loss of sensation, numbness, tingling, abnormal sensation (paresthesia), burning sensation, pain (neuralgia), decreased sensation, and/or an inability to determine joint position sense in an area, such as the limbs, or elsewhere. For example, a subject may experience numbness in the fingers and/or toes. Sensations often will begin in the feet or hands and progress towards the center of the body. Sensory peripheral neuropathy may result from the degeneration of the axon portion of a nerve cell, or the loss of the myelin sheath that may surround the axon of a nerve cell.

Motor neuropathies are another category of peripheral neuropathies. Motor peripheral neuropathy typically involves a dysfunction or damage to motor fibers that may impair the movement or function of an area supplied by a nerve because impulses to the area are blocked. Impaired nervous stimulation to a muscle group may result in weakness, decreased movement, decrease or lack of control of movement, difficulty or inability to move a part of the body (paralysis), muscle function or feeling loss, muscle atrophy, foot pain, or muscle twitching (fasciculation). This dysfunction typically manifests itself as a clumsiness in performing physical tasks or as muscular weakness. For example, patients may experience difficulty in buttoning a shirt or combing their hair. Muscular weakness may cause patients to become exhausted after relatively minor exertion and, in some cases, may create difficulty in standing or walking.

Structural changes in muscle, bone, skin, hair, nails, and body organs can also result from loss of nerve function, lack of nervous stimulation, not using an affected area, immobility, or lack of weight bearing. Peripheral motor neuropathy may manifest in a subject as muscle wasting or atrophy (loss of muscle mass).

Motor neuropathies often include many acquired primary demyelinating neuropathies such as Guillain-Barre syndrome. Other proximal symmetric motor polyneuropathies may be caused by chronic inflammatory demyelinating polyradiculoneuropathy (CIDP); diabetes mellitus; porphyria; osteosclerotic myeloma, Waldenstrom's macroglobulinemia; Castleman's disease; monoclonal gammopathy of undetermined significance; acute arsenic polyneuropathy; lymphoma; diphtheria; HIV/AIDS; Lyme disease; hypothyroidism; and vincristine toxicity. Demyelinating peripheral neuropathies include but are not limited to CIDP, osteosclerotic myeloma, diptheria, perhexilene toxicity, chloroquine toxicity, FK506 (tacrolimus) toxicity, procainamide toxicity, zimeldine toxicity, monoclonal protein-associated peripheral neuropathy, hereditary motor and sensory peripheral neuropathies types 1 and 3, and hereditary susceptibility to pressure palsies.

Motor neuropathies can also occur in Motor Neuron Diseases (MND) because MND can involve damage to peripheral motor neurons. MND include a group of severe disorders of the nervous system characterized by the progressive degeneration of motor neurons without sensory abnormalities. MND may affect the upper motor neurons, which are the nerves that lead from the brain to the spinal cord; the lower motor neurons, which are nerves that lead from the spinal cord to the muscles of the body; or both upper and lower motor neurons. Damage to the upper motor neurons is indicated by spasms, exaggerated reflexes, and extensor planter signs. Damage to the lower motor neurons is indicated by a progressive wasting (atrophy) and weakness of muscles that have lost their nerve supply. Human MND are characterized by paralysis, as well as a variety of other motor signs. MND include, but are not limited to Amyotrophic Lateral Sclerosis (ALS; Lou Gehrig's Disease), Progressive Bulbar Palsy, Spinal Muscular Atrophy (all types), Kugelberg-Welander Syndrome, Duchenne's Paralysis, post polio syndrome, Werdnig-Hoffman Disease, Kennedy's Disease, Juvenile Spinal Muscular Atrophy, Benign Focal Amyotrophy, and Infantile Spinal Muscular Atrophy.

In most cases of MND, degeneration in both the upper and lower motor neurons occurs. For example, ALS is characterized by muscle weakness, stiffness, and fasciculations (muscle twitching). In Progressive Bulbar Palsy, the muscles involving speech and swallowing are solely affected. Less common forms of MND involve the selective degeneration of either upper motor neurons (such as Primary Lateral Sclerosis) or lower motor neurons (Progressive Muscular Atrophy). There is considerable overlap between these forms of MND. R(−)DMS, S(+)DMS, or a combination of R(−)DMS and S(+)DMS can be used to treat MND, whether the disease involves upper motor neurons, lower motor neurons, or both upper and lower motor neurons.

Sensorimotor neuropathies are another class of peripheral neuropathies. Sensorimotor neuropathies involve both sensory and motor neurons, and typically denote a mixed nerve with afferent and efferent fibers. Many toxic and metabolic peripheral neuropathies present as a distal symmetric or dying-back process. Distal symmetric sensorimotor polyneuropathies may be due to endocrine diseases such as diabetes mellitus, hypothyroidism, and acromegaly; nutritional diseases such as alcoholism, vitamin B₁₂ deficiency, folate deficiency, Whipple's disease, thiamine deficiency, gastric restriction, and postgastrectomy; infectious diseases such as HIV and Lyme disease; connective tissue diseases such as rheumatoid arthritis, polyarteritis nodosa, systemic lupus, erythematosus, Churg-Strauss vasculitis, and cryoglobulinemia; toxic neuropathy by acrylamide, carbon disulfide, dichlorophenoxyacetic acid, ethylene oxide, hexacarbons, carbon monoxide, organophosphorous esters, or glue sniffing; medications such as vincristine, paclitaxel, nitrous oxide, colchicines, isoniazid, amitriptyline, ethambutol, disulfiram, cimetidine, phenytoin, dapsone, alfa interferon, lithium, didanosine, pyridoxine, metronidazole, hydralazine, cisplatin, thalidomide, pyridoxine, amiodarone, chloroquine, suramin, or gold; hypophosphatemia; carcinomatous axonal sensorimotor polyneuropathy; lymphomatous axonal sensorimotor polyneuropathy; sarcoidosis; amyloidosis; gouty neuropathy; or metal neuropathy by chronic arsenic intoxication, mercury, gold, or thallium.

The autonomic nervous system is the part of the peripheral nervous system that controls involuntary or semi-voluntary functions, such as the control of internal organs. The autonomic nervous system, also designated the visceral motor system, includes neurons that relay motor outflow to cardiac muscle, smooth muscle, and glands. The autonomic nervous system is commonly divided into two parts: the parasympathetic division and the sympathetic division; the functional activities of the two divisions generally oppose one another. For example, the parasympathetic division controls functions that will increase heart rate, while the sympathetic division generally functions to decrease heart rate.

Autonomic peripheral neuropathy typically involves a dysfunction of peripheral autonomic nerves, which may cause changes in the functioning of organs, and may result in symptoms such as blurred vision, double vision, decreased ability or inability to sweat (anhidrosis), dizziness or fainting that is often associated with a fall in blood pressure (postural hypotension), decreased ability to regulate body temperature, heat intolerance, disturbances in stomach or bowel function such as nausea, vomiting, constipation, or diarrhea, feeling full after eating a small amount (early satiety), unintentional weight loss (more than 5% of body weight), abdominal bloating, disturbances in bladder function (e.g., urinary incontinence or difficulty beginning to urinate), sexual dysfunction (e.g., male impotence), cardiac irregularities, and other toxicities.

Diabetes mellitus (also referred to hereinafter as “diabetes”), is a systemic disorder that primarily impacts the peripheral nervous system. Diabetes is also the most common cause of peripheral neuropathies. Virtually every individual who is diabetic for more than 10 to 15 years has some evidence of neuropathy. Virtually every aspect of the nervous system, including the central nervous system, as well as its supporting structures, can be affected by the complications of diabetes. Abnormally high concentrations of glucose in the circulating blood (called hyperglycemia) can be found in patients with diabetes. Diabetes is a significant risk factors for stroke, peripheral neuropathy, retinopathy, and nephropathy. Other complications associated with diabetes are diabetic ketoacidosis and coma, hyperosmolar nonketotic coma, chronic diabetic encephalopathy, cataract formation, and glaucoma.

Peripheral neuropathies are some of the most common complications of diabetes. These disorders are referred to as diabetic neuropathy. About two thirds of diabetic patients have one or more forms of diabetic peripheral neuropathy. Some of the symptoms of diabetic neuropathies are pain, which can be dull, burning, stabbing, crushing, or aching and cramplike; paresthesia, which may manifest as a sensation of coldness, numbness, tingling, or burning; and calf tenderness and pain. Peripheral neuropathies are generally divided into symmetric and asymmetric neuropathies. The majority of diabetic neuropathies present with predominant distal lower-limb involvement with symmetric sensorimotor polyneuropathies. Diabetic neuropathies can affect both sensory and motor peripheral nerves, as well as the autonomic nervous system.

Diabetic neuropathy can present as a small-fiber sensory neuropathy, often with early painful paresthesias, or a loss of pain and temperature sensation, with sparing of distal reflexes and proprioception. Diabetic neuropathic cachexia, which usually occurs after initiating insulin injections, is a severe form of painful diabetic neuropathy occurring in men. Diabetic neuropathy can also manifest as a large-fiber sensory neuropathy; autonomic neuropathy (involving both the sympathetic and parasympathetic nervous systems); motor neuropathy, also called diabetic amyotrophy; mixed polyneuropathy, for example a mixed sensory-autonomic-motor polyneuropathy; focal compression neuropathy; and truncal neuropathy. R(−)DMS, S(+)DMS, or a combination of R(−)DMS and S(+)DMS can be used to treat patients with any of the manifestations of diabetic neuropathy.

Chronic alcoholics may suffer from a peripheral neuropathy that is often painful. The main symptoms of alcoholic peripheral neuropathy (or alcoholic polyneuropathy) are burning, stabbing pains, and numbness in feet and hands. Sensory loss is often combined with painful hypersensitivity in the feet, loss of ankle reflexes, and mild distal weakness. Alcoholic peripheral neuropathy may be caused by the toxic effects of ethanol, malnutrition, or both. Distal, painful peripheral neuropathy is also common in the late stages of HIV infection. The main symptom of this peripheral neuropathy is continuous burning discomfort, usually in the feet, with some degree of sensory loss; motor involvement is usually minor. Acute and chronic inflammatory demyelinating peripheral neuropathies may also occur in otherwise asymptomatic people infected with HIV. R(−)DMS, S(+)DMS, or a combination of R(−)DMS and S(+)DMS can be used to treat patients with alcoholic polyneuropathy, as well as patients infected with HIV and suffering from peripheral neuropathy.

Subjects with certain systemic vasculitides also frequently suffer from peripheral neuropathy. Typically, the cause of vasculitic peripheral neuropathy is ischemia, i.e., a consequence of the inflammation of nutrient vessels of nerves by the inflammatory process. Normally nerves receive a robust supply of blood, and are relatively resistant to ischemic injury. Therefore, the development of vasculitic peripheral neuropathy implies extensive vascular disease. Approximately 3 0% of patients with vasculitic peripheral neuropathy have a symmetric polyneuropathy, approximately 30% have an asymmetric polyneuropathy, and approximately 40% have multiple mononeuropathies. Vasculitic peripheral neuropathy is mostly found in the systemic vasculitides polyarteritis nodosa, rheumatoid vasculitis, Sjogren's syndrome, Wegener's granulomatosis, and Churg-Strauss syndrome.

Inflammatory Sensory Polyganglionopathy (ISP) is a syndrome that involves relatively pure sensory loss (particularly proprioception) and areflexia. Sensory symptoms of ISP may begin abruptly or may evolve slowly, and the sensory ataxia is often severe and disabling. The early well-described cases of ISP were paraneoplastic, and the possibility of an underlying malignancy, particularly small cell lung cancer, should be considered when ISP is diagnosed. Other associations with ISP have also been reported, for example an association with Sjogren's syndrome, in which infiltration of dorsal root ganglia by T-lymphocytes has been demonstrated. R(−)DMS, S(+)DMS, or a combination of R(−)DMS and S(+)DMS can be used to treat patients with vasculitic peripheral neuropathy, as well as ISP.

It has been estimated that approximately 5% of patients admitted to intensive care units may develop peripheral neuropathy, which may be severe. Prolonged ICU admission, sepsis, and organ system failure are features that are common to many documented cases. R(−)DMS, S(+)DMS, or a racemic mixture of the two can be used to treat patients in the ICU to prevent or treat peripheral neuropathy.

There are a number of causes of peripheral neuropathy, including but not limited to toxic agents such as chemotherapeutic agents, genetically inherited conditions, systemic diseases, and nerve destruction by trauma or pressure. Degeneration of an axon will slow or block conduction of impulses through the nerve at the point of the degeneration. Systemic causes of peripheral neuropathy include disorders that affect the connective tissues of the nerves or the blood supply to the nerves, as well as metabolic or chemical disorders, and other disorders that damage peripheral nerve tissue.

The particular systemic disease, localized disease, hereditary condition, toxic agent, or trauma responsible for causing peripheral neuropathy is not critical to the present disclosure. Thus, R(−)DMS, S(+)DMS, or a mixture of R(−)DMS and S(+)DMS, is effective for peripheral neuropathies associated with systemic diseases including but not limited to: acute inflammatory or immune-mediated peripheral neuropathies such as chronic inflammatory demyelinating polyradiculoneuropathy (CIDP), acute inflammatory demyelinating polyneuropathy (AIDP), Guillain-Barre syndrome, acute motor axonal neuropathy (AMAN), acute motor and sensory asonal neuropathy (AMSAN), Miller-Fisher syndrome, ganglioneuritis, and pandysautonomia; inflammatory plexopathies such as brachial plexitis and lumbosacral plexitis; infectious peripheral neuropathies such as herpes simplex infection, herpes zoster virus (shingles), hepatitis B, hepatitis C, acquired immunodeficiency syndrome (AIDS)—associated neuropathy, HIV infection, cytomegalovirus infection, Colorado tick fever, diphtheria, syphilis, leprosy, trypanosoma cruzi (Chagas' disease), Lyme disease, Campylobacter jejuni infection, and poliomyelitis; uremia; botulism; childhood cholestatic liver disease; chronic respiratory insufficiency; alcoholic neuropathy; multiple organ failure; sepsis; hypo-albuminemia; eosinophilia-myalgia syndrome; porphyria; hypo-glycemia; chronic gluten enteropathy; vitamin deficiency; dietary deficiency (e.g. vitamin B₁₂ deficiency; thiamine deficiency (beriberi); vitamin E deficiency; folate deficiency); Whipple's disease; postgastrectomy syndrome; iron deficiency; chronic liver disease; primary biliary cirrhosis; hypophosphatemia; hyperlipidemia; Waldenstrom's macroglobulinemia; tabes dorsalis; Crohn's disease; atherosclerosis; Gouty neuropathy; sensory perineuritis; Sjögren's syndrome; primary vasculitis (such as polyarteritis nodosa); Churg-Strauss vasculitis; allergic granulomatous angiitis; hypersensitivity angiitis; Wegener's granulomatosis; rheumatoid arthritis; myxedema; Inflammatory Sensory Polyganglionopathy (ISP); systemic lupus erythematosis; mixed connective tissue disease; scleroderma; sarcoidosis; vasculitis; systemic vasculitides; acute tunnel syndrome; carcinomatous axonal sensorimotor polyneuropathy; lymphomatous axonal sensorimotor polyneuropathy; primary, secondary, localized or familial systemic amyloidosis; hypothyroidism; carpal tunnel syndrome; sciatica; chronic obstructive pulmonary disease; acromegaly; malabsorption (sprue, celiac disease); carcinomas (sensory, sensorimotor, late, and demyelinating); lymphoma (including Hodgkin's), polycythemia vera; multiple myeloma (lytic type, osteosclerotic, or solitary plasmacytoma); lymphomatoid granulomatosis; benign monoclonal gammopathy; lung cancer; leukemia; macroglobulinemia; cryoglobulinemia; tropical myeloneuropathies; diabetes mellitus; and diabetic amyotrophy. Peripheral neuropathies are also associated with mitochondrial diseases. A significant percentage of peripheral neuropathies are idiopathic, and R(−)DMS, S(+)DMS, or a racemic mixture of the two can also be used to prevent or treat these peripheral neuropathies.

Genetically acquired peripheral neuropathies suitable for treatment by R(−)DMS, S(+)DMS, or a combination thereof include, without limitation: peroneal muscular atrophy (Charcot-Marie-Tooth Disease) hereditary amyloid neuropathies, hereditary sensory neuropathy (type I and type II), porphyric neuropathy, hereditary liability to pressure palsy, congenital hypomyelinating neuropathy, familial brachial plexus neuropathy, porphyries, Fabry's Disease, adrenomyeloneuropathy, Riley-Day Syndrome, Dejerine-Sottas neuropathy (hereditary motor-sensory neuropathy-III), Refsum's disease, ataxia-telangiectasia, hereditary tyrosinemia, anaphalipoproteinemia, abetalipoproteinemia, giant axonal neuropathy, metachromatic leukodystrophy and adrenoleukodystrophy, globoid cell leukodystrophy, and Friedrich's ataxia.

R(−)DMS, S(+)DMS, or a combination of R(−)DMS and S(+)DMS may also be used to treat peripheral neuropathy caused by a toxic agent. Toxins that produce peripheral neuropathy can generally be divided into three groups: drugs and medications; industrial chemicals; and environmental toxins. As used herein, the term “toxic agent” is defined as any substance that, through its chemical action, impairs the normal function of one or more components of the peripheral nervous system. The definition includes agents that are airborne, ingested as a contaminant of food or drugs, or taken deliberately as part of a therapeutic regime.

The list of toxic agents that may cause peripheral neuropathy includes, but is not limited to, acetazolamide, acrylamide, adriamycin, alcohol, allyl chloride, almitrine, amitriptyline, amiodarone, amphotericin, arsenic, aurothioglucose, carbamates, carbon disulfide, carbon monoxide, carboplatin, chloramphenicol, chloroquine, cholestyramine, cimetidine, cisplatin, cis-platinum, clioquinol, colestipol, colchicine, colistin, cycloserine, cytarabine, dapsone, dichlorophenoxyacetic acid, didanosine; dideoxycytidine, dideoxyinosine, dideoxythymidine, dimethylaminopropionitrile, disulfiram, docetaxel, doxorubicin, ethambutol, ethionamide, ethylene oxide, FK506 (tacrolimus), glutethimide, gold, hexacarbons, hexane, hormonal contraceptives, hexamethylolmelamine, hydralazine, hydroxychloroquine, imipramine, indomethacin, inorganic lead, inorganic mercury, isoniazid, lithium, methylmercury, metformin, methylbromide, methylhydrazine, metronidazole, misonidazole, methyl N-butyl ketone, nitrofurantoin, nitrogen mustard, nitrous oxide, organophosphates, ospolot, paclitaxel, penicillin, perhexiline, perhexiline maleate, phenytoin, platinum, polychlorinated biphenyls, primidone, procainamide, procarbazine, pyridoxine, simvastatin, sodium cyanate, streptomycin, sulphonamides, suramin, tamoxifen, thalidomide, thallium, toluene, triamterene, trimethyltin, triorthocresyl phosphate, L-tryptophan, vacor, vinca alkaloids, vindesine, megadoses of vitamin A, megadoses of vitamin D, zalcitamine, zimeldine; industrial agents, especially solvents; heavy metals; and sniffing glue or other toxic compounds. Other peripheral neuropathies that may be treated by the present disclosure include neuropathies due to ischemia or prolonged exposure to cold temperatures.

Although the particular disease, toxic agent, or trauma causing the peripheral neuropathy is not critical, the present disclosure will be particularly valuable in the treatment of peripheral neuropathy resulting from the administration of chemotherapeutic agents to cancer patients. Among the chemotherapeutics known to cause peripheral neuropathy are vincristine, vinblastine, cisplatin, paclitaxel, procarbazine, dideoxyinosine, cytarabine, alpha interferon, and 5-fluorouracil (see Macdonald, Neurologic Clinics 9: 955-967 (1991)).

As stated, the present disclosure encompasses the treatment of peripheral neuropathy, including the prevention, alleviation, reduction, or elimination, in whole or in part, of symptoms associated with peripheral neuropathy, by use of DMS in the form of R(−)DMS, S(+)DMS, or mixtures of R(−)DMS and S(+)DMS. As used herein, the term R(−)DMS means the R(−) enantiomeric form of DMS, including as a free base, as well as any acid addition salt thereof. Likewise, the term S(+)DMS, as used herein, encompasses the S(+) enantiomeric form of DMS, including as a free base, as well as any acid addition salt thereof. Such salts of either R(−)DMS or S(+)DMS include those derived from organic and inorganic acids such as, without limitation, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embonic acid, enanthic acid, and the like. Accordingly, reference herein to the administration of either or both R(−)DMS and S(+)DMS encompasses both the free base and acid addition salt forms. When either R(−)DMS or S(+)DMS is used alone in the presently disclosed compositions and methods, it is used in a substantially enantiomerically pure form. Reference to mixtures or combinations of R(−)DMS and S(+)DMS includes both racemic and non-racemic mixtures of optical isomers.

R(−)DMS and/or S(+)DMS may be administered either by an oral route (involving gastrointestinal absorption) or by a non-oral route (does not rely upon gastrointestinal absorption, i.e. a route that avoids absorption of R(−)DMS and/or S(+)DMS from the gastrointestinal tract). Depending upon the particular route employed, the DMS is administered in the form of the free base or as a physiologically acceptable non-toxic acid addition salt as described above. The use of salts, especially the hydrochloride, is particularly desirable when the route of administration employs aqueous solutions, as for example parenteral administration; use of delivered desmethylselegiline in the form of the free base is especially useful for transdermal administration. Although the oral route of administration will generally be most convenient, R(−)DMS, S(+)DMS, or a mixture of both may be administered by oral, peroral, enteral, pulmonary, nasal, lingual, intravenous, intraarterial, intracardial, intramuscular, intraperitoneal, intracutaneous, subcutaneous, parenteral, topical, transdermal, intraocular, buccal, sublingual, intranasal, inhalation, vaginal, rectal, or other routes as well.

The optimal daily dose of R(−)DMS, S(+)DMS, or of a combination of the two, such as a racemic mixture of R(−)DMS and S(+)DMS, useful for the purposes of the present invention is determined by methods known in the art, e.g., based on the severity of the peripheral neuropathy and symptoms being treated, the condition of the subject to whom treatment is being given, the desired degree of therapeutic response, and the concomitant therapies being administered to the patient or animal. The total daily dosage administered to a patient, typically a human patient, should be at least the amount required to prevent, reduce, or eliminate one or more of the symptoms associated with peripheral neuropathy, typically one of the symptoms discussed above.

Ordinarily, the attending physician will administer an initial daily non-oral dose of at least about 0.01 mg per kg of body weight, calculated on the basis of the free secondary amine, with progressively higher doses being employed depending upon the response to therapy. The final daily dose will be between about 0.05 mg/kg of body weight and about 0.15 mg/kg of body weight (all such doses again being calculated on the basis of the free secondary amine). Ordinarily, however, the attending physician or veterinarian will administer an initial dose of at least about 0.015 mg/kg, calculated on the basis of the free secondary amine, with progressively higher doses being employed depending upon the route of administration and the subsequent response to the therapy. Typically the daily dose will be from about 0.02 mg/kg or 0.05 mg/kg to about 0.10 mg/kg or about 0.15 mg/kg to about 0.175 mg/kg or about 0.20 mg/kg or about 0.5 mg/kg and may extend to about 1.0 mg/kg or even 1.5, 2.0, 3.0 or 5.0 mg/kg of the patient's body weight depending on the route of administration. Preferred daily doses will be in the range of about 0.10 mg/kg to about 1.0 mg/kg. More preferred daily doses will be in the range of about 0.4 mg/kg to about 0.9 mg/kg. Even more preferred daily doses will be in the range of about 0.6 mg/kg to about 0.8 mg/kg. Again, all such doses should be calculated on the basis of the free secondary amine. In other preferred embodiments, the daily dose will be in the range of about 0.01 mg to about 1000 mg per day. Preferred doses will be about 0.05, 0.1, 0.2, 0.3, 0.4, 0.5, 1.0, 2.0, 3.0, 4.0, 5.0, 10, 20, 30, 40, 50, 60, 70, 80, 90, 100, 150, 200, 300, 400, 500, 600, 700, 800, 900, or 1000 mg per day.

These are simply guidelines since the actual dose must be carefully selected and titrated by the attending physician based upon clinical conditions. The optimal daily dose will be determined by methods known in the art and will be influenced by factors such as the age and weight of the patient, the clinical condition of the patient, the condition or disease associated with the peripheral neuropathy, the severity of both the peripheral neuropathy and the disease, the condition of the patient to whom treatment is being given, the desired degree of therapeutic response, the concomitant therapies being administered, and observed response of the individual patient or animal. The daily dose can be administered in a single or multiple dosage regimen.

Either oral or non-oral dosage forms may be used and may permit, for example, a burst of the active ingredient from a single dosage unit, such as an oral composition or sublingual or buccal administration, or a continuous release of relatively small amounts of the active ingredient from a single dosage unit, such as a transdermal patch, over the course of one or more days. Alternatively, intravenous or inhalation routes may be preferred. A number of different dosage forms may be used to administer the R(−)DMS, S(+)DMS, or a combination of R(−)DMS and S(+)DMS, including but not limited to tablets, pills, capsules, powders, aerosols, suppositories, skin patches, parenterals, and oral liquids, include oil aqueous suspensions, solutions, and emulsions. Additionally, desmethylselegiline-containing sustained release (long acting) formulations and devices are contemplated.

Pharmaceutical compositions containing one or both R(−)DMS or S(+)DMS can be prepared according to conventional techniques. For example, preparations for parenteral routes of administration, e.g., intramuscular, intravenous, intrathecal, and intraarterial routes, can employ sterile isotonic saline solutions. Sterile buffered solutions can also be employed for intraocular administration.

Transdermal dosage unit forms of R(−)DMS and/or S(+)DMS can be prepared utilizing a variety of previously described techniques (see e.g., U.S. Pat. Nos. 4,861,800; 4,868,218; 5,128,145; 5,190,763; and 5,242,950; and EP-A 404807, EP-A 509761, and EP-A 593807, incorporated herein by reference). For example, a monolithic patch structure can be utilized in which desmethylselegiline is directly incorporated into the adhesive and this mixture is cast onto a backing sheet. Alternatively R(−)DMS and/or S(+)DMS, can be incorporated as an acid addition salt into a multilayer patch which effects a conversion of the salt to the free base, as described for example in EP-A 593807 (incorporated herein by reference). Specifically contemplated by the present disclosure is a transdermal patch composition that has about 5 mg, 10 mg, 20 mg, 30 mg, 50 mg, or 100 mg of R(−)DMS, S(+)DMS, or a combination of R(−)DMS and S(+)DMS.

One or both R(−)DMS or S(+)DMS can also be administered by a device employing a lyotropic liquid crystalline composition in which, for example, 5 to 15% of desmethylselegiline is combined with a mixture of liquid and solid polyethylene glycols, a polymer, and a nonionic surfactant, optionally with the addition of propylene glycol and an emulsifying agent. For further details on the preparation of such transdermal preparations, reference can be made to EP-A 5509761 (incorporated herein by reference). Additionally, buccal and sublingual dosage forms of R(−)DMS, S(+)DMS, or a combination of R(−)DMS and S(+)DMS may be prepared utilizing techniques described in, for example, U.S. Pat. Nos. 5,192,550; 5,221,536; 5,266,332; 5,057,321; 5,446,070; 4,826,875; 5,304,379; or 5,354,885 (incorporated herein by reference).

Subjects treatable by the present preparations and methods include both human and non-human subjects. Accordingly, the compositions and methods above provide especially useful therapies for mammals, including humans, and in domesticated mammals. Thus, the present methods and compositions are used in treating peripheral neuropathy in human, primate, canine, feline, bovine, equine, ovine, murine, caprine, and porcine species, and the like.

Treatment by the administration of R(−)DMS, S(+)DMS, or a combination of R(−)DMS and S(+)DMS should be continued until the symptoms associated with peripheral neuropathy subside. The drug may be either administered at regular intervals (e.g., twice a day) or delivered in an essentially continuous manner, e.g., via a transdermal patch. Patients should be regularly evaluated by physicians, e.g. once a week, once a month, twice a year, etc., to determine whether there has been an improvement in symptoms and whether the dosage of desmethylselegiline needs to be adjusted. Since delayed progressive peripheral neuropathy has been demonstrated after the cessation of cisplatin therapy (see e.g. Grunberg et al., Cancer Chemother. Pharmacol. 25:62-64 (1989)), it is preferred that administration of R(−)DMS, S(+)DMS, or a combination of the two be continued for a period (e.g. from about 1-12 months) after the end of chemotherapy. Additionally, the administration of R(−)DMS, S(+)DMS, or a combination of the two may be used to prevent the onset of symptoms associated with peripheral neuropathy, particularly when a subject is at risk for developing peripheral neuropathy.

The present disclosure is also directed to a method for treating cancer patients that are being treated with a chemotherapeutic agent known to cause peripheral neuropathy by using a combination of chemotherapeutic agent and R(−)DMS, S(+)DMS, or a mixture of R(−)DMS and S(+)DMS. Except as noted below, the same considerations discussed in the sections above apply equally to the situation in which R(−)DMS, S(+)DMS, or a combination of the two is used as part of a therapeutic regime for such patients.

R(−)DMS, S(+)DMS, or a racemic mixture of R(−)DMS and S(+)DMS may be used in combination with any chemotherapeutic agent that causes peripheral neuropathy as a side effect. Treatment is especially preferred for chemotherapeutic agents that are so toxic that their dosage is limited by the peripheral neuropathy that they cause. Included in this group are paclitaxel, cisplatin, vincristine, and vinblastine. By preventing or reducing the peripheral neuropathy associated with these agents, R(−)DMS, S(+)DMS, or a combination of the two allows higher individual doses to be administered to patients, thereby increasing the overall efficacy of the therapy. Additionally, the administration of R(−)DMS, S(+)DMS, or a combination of the two allows patients to receive a higher cumulative dose of chemotherapeutic agent. Increased cumulative dose may result from higher doses of the chemotherapeutic agent being administered at each therapeutic cycle, an increase in the number of cycles, or a combination of higher doses and more cycles.

The most preferred chemotherapeutic agents for use in the present disclosure are cisplatin and paclitaxel, both of which are severely toxic to peripheral nerves, which limits the dosages that maybe safely administered to a patient (see Macdonald, Neurologic Clinics 9: 955-967 (1991)). Although dose intensity of these agents is an important factor in achieving optimal therapeutic results, doses substantially above about 75-100 mg/m² for cisplatin (Ozols, Seminars in Oncology 16: 22-30 (1989)) and about 175-225 mg/m² for paclitaxel (Gianni, et al., J. Nat'l Cancer Inst. 87:1169-75 (1995)), typically cannot be given.

The symptoms associated with peripheral neuropathy caused by the administration of cisplatin include sensory polyneuropathy with paresthesias, vibratory and proprioceptive loss, loss of pain and temperature sensation, and reduced deep tendon reflexes (see Macdonald, Neurologic Clinics 9:955-967 (1991); Ozols, Seminars in Oncology 16, suppl. 6:22-30 (1989)). Symptoms associated with other agents such as vincristine and paclitaxel include loss of deep tendon reflex response at the ankle which may progress to complete areflexia, distal symmetric sensory loss, motor weakness, foot drop, muscle atrophy, constipation, ileus, urinary retention, impotence, and postural hypotension (Id.; Casey, et al., Brain 96: 69-86 (1973)). For the purposes of the present disclosure, the severity of these symptoms is considered to be unacceptable when either a patient judges them to be intolerable or the patient's physician judges them to pose so serious a threat to the patient's health that the dosage of chemotherapeutic agent must be reduced or discontinued.

The particular route of administration of R(−)DMS, S(+)DMS, or a mixture of R(−)DMS and S(+)DMS that is most preferred for a patient treated with a chemotherapeutic agent will be determined by clinical considerations and may include any of the routes of delivery or dosage forms discussed above. Routes of administration which avoid gastrointestinal absorption may be preferred. Thus, preferred routes will typically include transdermal, parenteral, sublingual, and buccal administration.

In some instances, patients administered R(−)DMS, S(+)DMS, or a combination of R(−)DMS and S(+)DMS according to the present disclosure will already have been on chemotherapy at the time that R(−)DMS, S(+)DMS, or a mixture of R(−)DMS and S(+)DMS treatment is initiated. As a result, an upper limit on the dosage of the chemotherapeutic agent may already have been established, beyond which the patient experiences unacceptably severe peripheral neuropathy. In these cases, administration of the chemotherapeutic agent should be maintained and treatment with R(−)DMS, S(+)DMS, or a combination of R(−)DMS and S(+)DMS initiated. The exact time at which chemotherapeutic and R(−)DMS, S(+)DMS, or a combination of the two are given relative to one another is not critical, provided that their therapeutic effects overlap. For example, it is not essential that the chemotherapeutic agent and R(−)DMS, S(+)DMS, or a combination of the two be administered in a single dosage form or within an hour or two of one another.

In instances in which a subject is taking multiple drugs or in which there is some reason to believe that they may be unusually sensitive to R(−)DMS, S(+)DMS, or a combination of the two, it may be desirable to start with a low initial dose (e.g., 0.01 mg/kg) in order to ensure that the subject is able to tolerate the medication. Once this is established, the dosage maybe adjusted upward. The effect of R(−)DMS, S(+)DMS, or a combination of the two on the symptoms of peripheral neuropathy should be evaluated by the subject over a period of time and by the subject's physician on a regular basis. Once a concentration of R(−)DMS, S(+)DMS, or a combination of the two is established that is effective at reducing symptoms, the dosage of the chemotherapeutic agent is increased until a new upper limit is established, i.e. until a dosage is established that cannot be exceeded without causing unacceptable side effects. The administration of R(−)DMS, S(+)DMS, or a combination of the R(−)DMS and S(+)DMS should be continued for a period oftime after the administration of the chemotherapeutic agent has ceased in order to prevent delayed and progressive peripheral neuropathy. For example, the subject may continue to receive R(−)DMS, S(+)DMS, or a combination of the two for a month or more after the end of chemotherapy.

The same basic procedure described above can be used for subjects beginning chemotherapy. In these cases, both the dosage of chemotherapeutic agent and R(−)DMS, S(+)DMS, or a combination of the two will have to be established. The preferred procedure is to begin by pretreating patients with R(−)DMS, S(+)DMS, or a combination of the two before the administration of the chemotherapeutic agent is begun. For example, a subject may be given 10 mg of R(−)DMS, S(+)DMS, or a combination of the two per day for a period of one week before treatment with the chemotherapeutic agent is initiated. The dosages of both the chemotherapeutic agent and R(−)DMS, S(+)DMS, or a combination of the two are then optimized as described above. Again, R(−)DMS, S(+)DMS, or a combination of R(−)DMS and S(+)DMS administration should be continued after the administration of the chemotherapeutic agent has stopped.

The present disclosure further encompasses methods for treating peripheral neuropathy by administering to the patient a pharmaceutical composition that includes R(−)DMS, S(+)DMS, or combinations of the two (which are conveniently prepared by methods known in the art, as described in Example 1) and one or more additional therapeutic agents known to treat peripheral neuropathy. Therapeutic agents known to treat the symptoms of peripheral neuropathy in various disorders include, but are not limited to, prednisone, IVIg, cyclophosphamide, famciclovir, tegretol, tricyclic antidepressants, dapsone, clofazamine, rifampin, nifurtimox, benznidaxole, gabapentin, ganciclovir, foscarnet, cidofovir, acyclovir, topical Lidocaine, and ribavirin. Such a pharmaceutical composition may be used to prevent or treat peripheral neuropathy. The therapeutic agents used in combination with R(−)DMS, S(+)DMS, or a mixture of the two to treat a peripheral neuropathy can also be presented to the patient in separate formulations. Thus, separate administration of a therapeutic agent or even administration that is spaced in time is contemplated by the present disclosure, particularly when the therapeutic agent and the DMS enantiomer or DMS enantiomers have a synergistic therapeutic action.

Successful use of the compositions and methods above requires employment of a therapeutically effective amount of R(−)DMS, S(+)DMS, or combination of R(−)DMS and S(+)DMS. As described above and notwithstanding its demonstrably inferior inhibitory properties with respect to MAO-B inhibition, R(−)DMS and its enantiomer appear to be at least if not more effective than selegiline for treating peripheral neuropathy.

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The following working examples are illustrative only, and are not intended to limit the scope of the invention.

### EXAMPLE 1

### Preparation of R(−)DMS and S(+)DMS

A. R(−)-desmethylselegiline

R(−)DMS is prepared by methods known in the art. For example, desmethylselegiline is a known chemical intermediate for the preparation of selegiline as described in U.S. Pat. No. 4,925,878. Desmethylselegiline can be prepared by treating a solution of R(−)-2-aminophenylpropane (levoamphetamine):

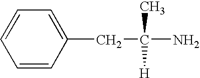

in an inert organic solvent such as toluene with an equimolar amount of a reactive propargyl halide such as propargyl bromide, Br—CH₂—C≡—CH, at slightly elevated temperatures (70°-90° C.). Optionally the reaction can be conducted in the presence of an acid acceptor such as potassium carbonate. The reaction mixture is then extracted with aqueous acid, for example 5% hydrochloric acid, and the extracts are rendered alkaline. The nonaqueous layer which forms is separated and extracted with for example, benzene, distilled, and dried under reduced pressure.

Alternatively the propargylation can be conducted in a two-phase system of a water-immiscible solvent and aqueous alkali, utilizing a salt of R(+)-2-aminophenylpropane with a weak acid such as the tartrate, analogously to the preparation of selegiline as described in U.S. Pat. No. 4,564,706.

B. S(+)-desmethylselegiline

S(+)DMS is conveniently prepared from the enantiomeric S(+)-2-aminophenylpropane (dextroamphetamine), i.e.,

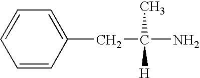

following the procedures set forth above for desmethylselegiline.

C. Mixtures of Enantiomers

Mixtures of the R(−) and S(+) enantiomeric forms of desmethylselegiline, including racemic desmethylselegiline, are conveniently prepared from enantiomeric mixtures, including racemic mixtures of the above aminophenylpropane starting material.

D. Conversion Into Acid Addition Salts

N-(prop-2-ynyl)-2-aminophenylpropane in either optically active or racemic form can be converted to a physiologically acceptable non-toxic acid addition salt by conventional techniques such as treatment with a mineral acid. For example, hydrogen chloride in isopropanol is employed in the preparation of desmethylselegiline hydrochloride. Either the free base or salt can be further purified, again by conventional techniques such as recrystallization or chromatography.

### EXAMPLE 2

### Characteristics of Substantially Pure R(−)DMS

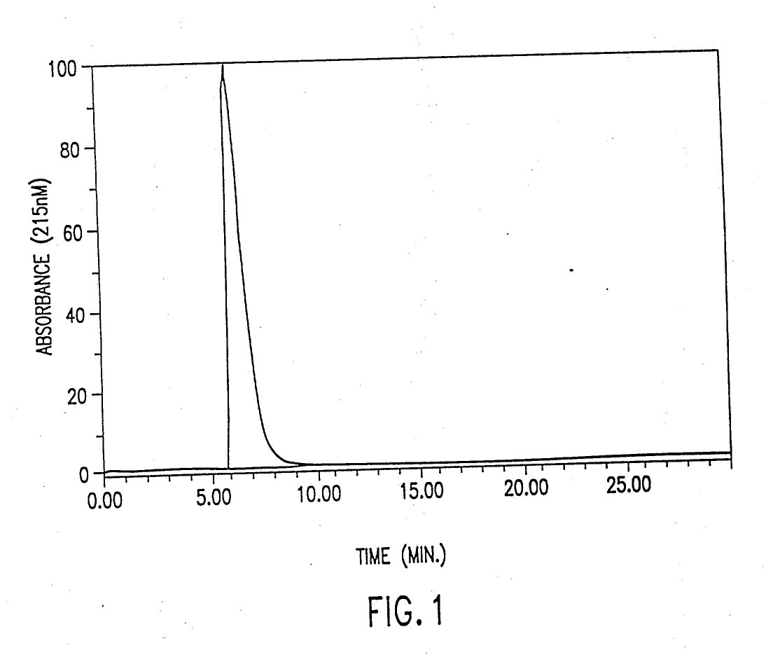

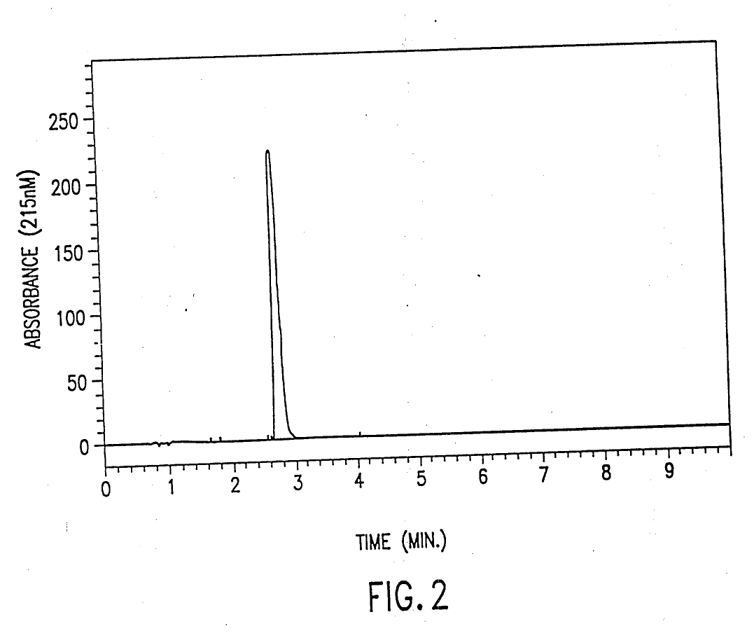

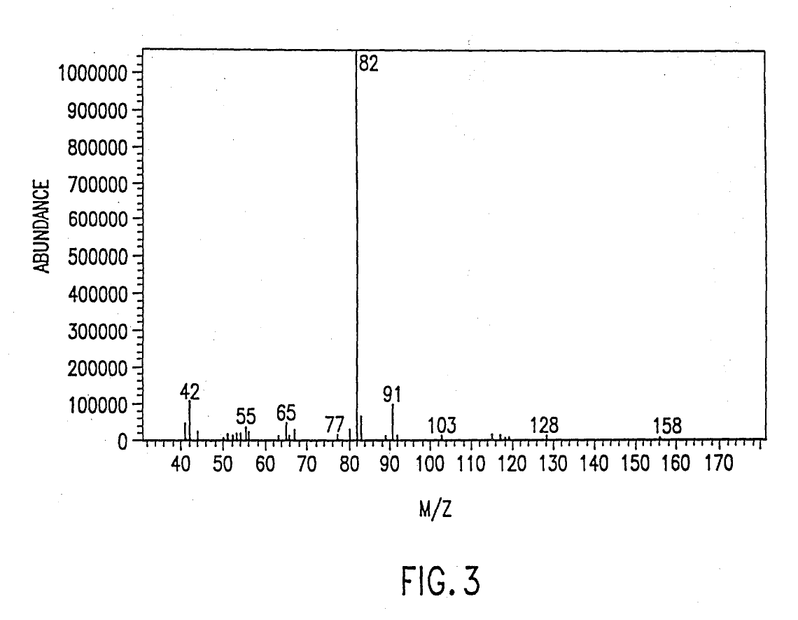

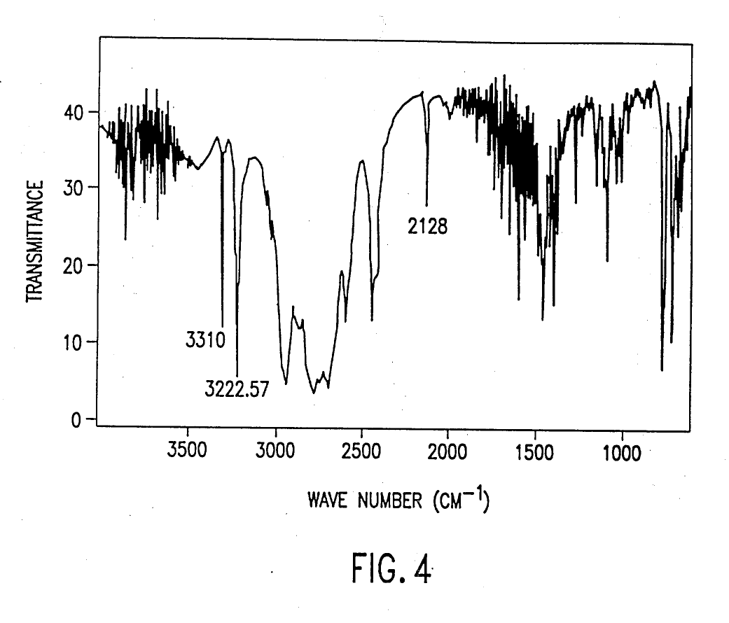

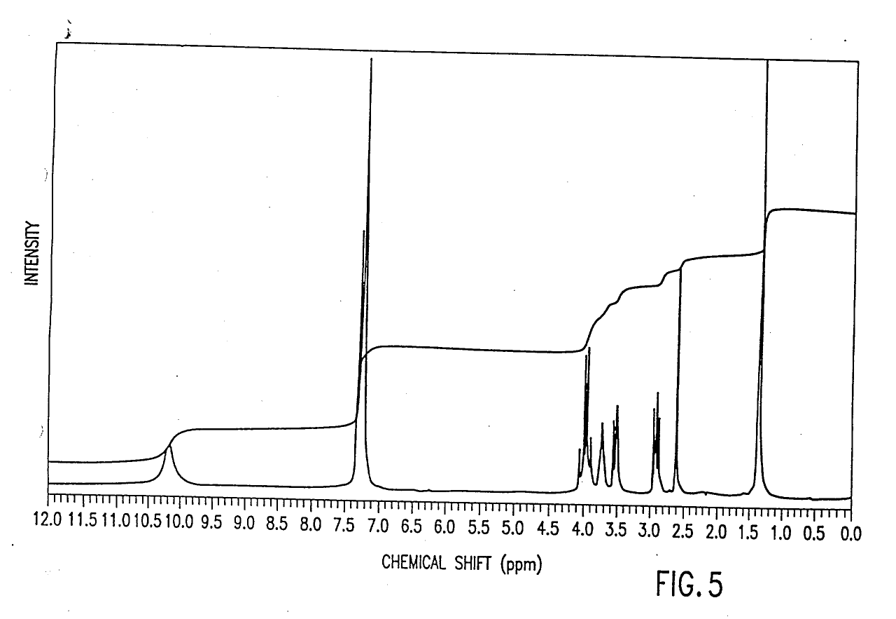

A preparation of substantially pure R(−)DMS has the appearance of a white crystalline solid with a melting point of 162-163 C. and an optical rotation of [α]_{D}^{23c}=−15.2±2.0 when measured at a concentration of 1.0 M using water as solvent. R(−)DMS appeared to be 99.5% pure when analyzed by HPLC on a Microsorb MV Cyano column (see chromatogram in FIG. 1) and 99.6% pure when analyzed by HPLC on a Zorbax Mac-Mod SB-C 18 column, (see chromatogram in FIG. 2). No single impurity is present at a concentration greater than or equal to 0.5%. Heavy metals are present at a concentration of less than 10 ppm and amphetamine hydrochloride at a concentration of less than 0.03%. The last solvents used for dissolving the preparation, ethyl acetate and ethanol are both present at a concentration of less than 0.1%. A mass spectrum performed on the preparation (see FIG. 3) is consistent with a compound having a molecular weight of 209.72 amu and a formula of C₁₂H₁₅N.HCl. Infrared and NMR spectra are shown in FIGS. 4 and 5 respectively. These are also consistent with the known structure of R-(−)-DMS.

### EXAMPLE 3

### Characteristics of Substantially Pure S(+)DMS

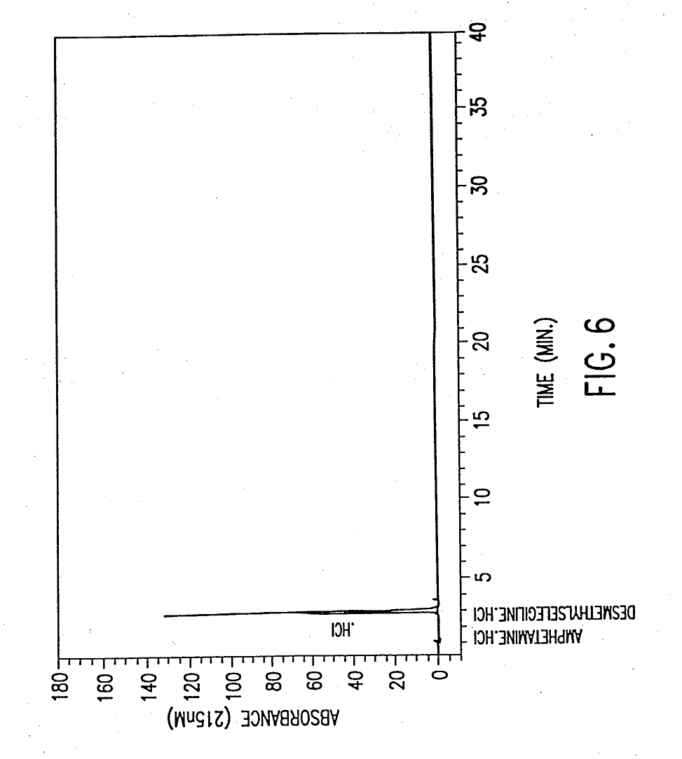

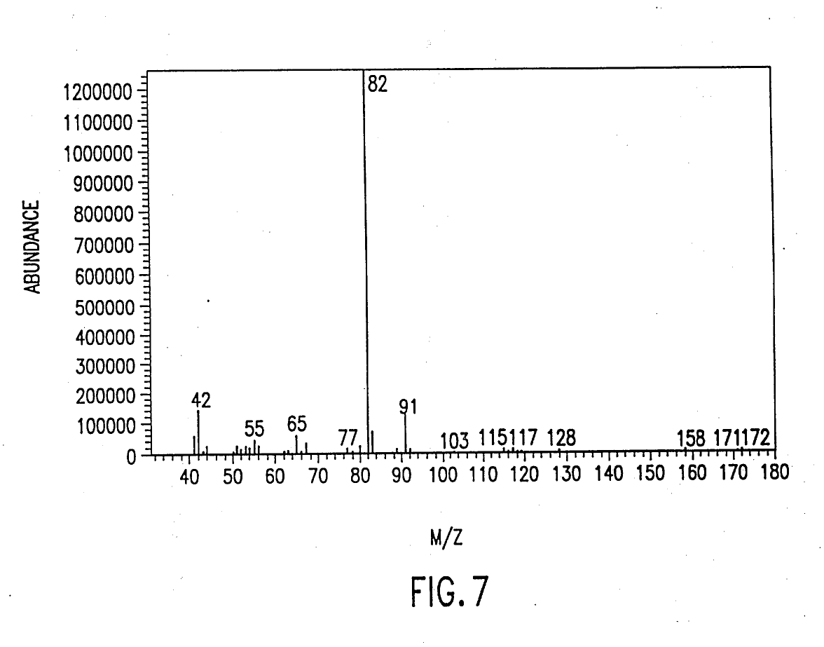

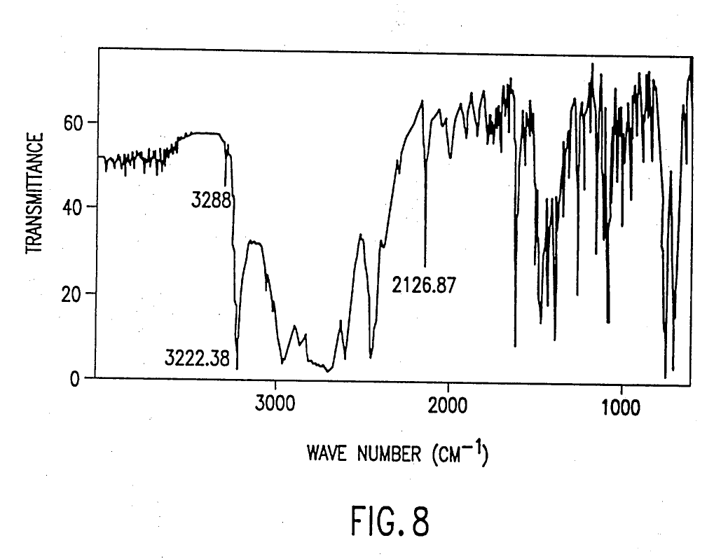

A preparation of substantially pure S(+)DMS has the appearance of a white powder with a melting point of approximately 160.04° C. and a specific rotation of +15.1 degrees when measured at 22° C. in water, at a concentration of 1.0 M. When examined by reverse phase HPLC on a Zorbax Mac-Mod SB-C18 column the preparation appears to be about 99.9% pure (FIG. 6). Amphetamine hydrochloride is present at a concentration of less than 0.13% (w/w). A mass spectrum is performed on the preparation and is consistent with a compound having a molecular weight of 209.72 and a molecular formula of C₁₂H₁₅N.HCI(see FIG. 7). Infrared spectroscopy is performed and also provides results consistent with the structure of S(+)DMS (see FIG. 8).

### EXAMPLE 4

### Actions of the R(−) and S(+) Enantiomers of Desmethylselegiline (DMS) on Human Platelet MAO-B and Guinea Pig Brain MAO-B and MAO-A Activity

Human platelet MAO is comprised exclusively of the type-B isoform of the enzyme. In the present study, the in vitro and in vivo inhibition of this enzyme by the two enantiomers of DMS was determined and compared with inhibition due to selegiline. The present study also examined the two enantiomers of DMS for inhibitory activity with respect to the MAO-A and MAO-B in guinea pig hippocampal tissue. Guinea pig brain tissue is an excellent animal model for studying brain dopamine metabolism, the enzyme kinetics of the multiple forms of MAO and the inhibitory properties of novel agents that interact with these enzymes. The multiple forms of MAO in this animal species show similar kinetic properties to those found in human brain tissue. Finally, the test agents were administered to guinea pigs and the extent to which they might act as inhibitors of brain MAO in vivo was assessed.

A. Method of Testing

In vitro: The test system utilized the in vitro conversion of specific substrates of MAO-A (¹⁴C-serotonin) in guinea pig hippocampal homogenates or MAO-B (¹⁴C-phenylethylamine) by human platelets and guinea pig hippocampal homogenates. The rate of conversion of each substrate was measured in the presence of S(+)DMS, R(−)DMS or selegiline and compared to the isozyme activity in the absence of these agents. A percent inhibition was calculated from these values. Potency was evaluated by comparing the concentration of each agent which caused a 50% inhibition(IC₅₀ value).

In vivo: R(−)DMS, S(+)DMS or selegiline was administered in vivo subcutaneously (sc), once a day for 5 days prior to sacrifice. Hippocampal homogenates containing enzyme were prepared, and assayed ex vitro for MAO-A and MAO-B activity. These experiments were performed to demonstrate that the DMS enantiomers were capable of entering brain tissue and inhibiting MAO activity.

B. Results

MAO-B Inhibitory Activity In Vitro

Results for MAO-B inhibition are shown in Tables 2 and 3. IC₅₀ values for MAO-B inhibition and potency as compared to selegiline is shown in Table 4.

TABLE 2
MAO-B Inhibition in Human Platelets Concentration

| | | |
|---|---|---|
| | | % Inhibition |
| Agent | Concentration | 0 ± SEM |

| | | | |
|---|---|---|---|
| Selegiline | 0.3 | nM | 8.3 ± 3.4 |
| | 5 | nM | 50.3 ± 8.7 |
| | 10 | nM | 69.0 ± 5.5 |
| | 30 | nM | 91.0 ± 1.4 |
| | 100 | nM | 96.0 ± 1.6 |
| | 300 | nM | 96.0 ± 1.6 |
| | 1 | μM | 96.6 ± 1.6 |
| R(−)DMS | 100 | nM | 14.3 ± 3.6 |
| | 300 | nM | 42.1 ± 4.0 |
| | 1 | μM | 76.9 ± 1.47 |
| | 3 | μM | 94.4 ± 1.4 |
| | 10 | μM | 95.8 ± 1.4 |
| | 3 | μM | 95.7 ± 2.3 |
| S(+)DMS | 300 | nM | 6.4 ± 2.8 |
| | 1 | μM | 11.1 ± 1.0 |
| | 3 | μM | 26.6 ± 1.9 |
| | 10 | μM | 42.3 ± 2.3 |
| | 30 | μM | 68.2 ± 2.34 |
| | 100 | μm | 83.7 ± 0.77 |
| | 1 | mM | 94.2 ± 1.36 |

TABLE 3
MAO-B Inhibition in Guinea Pig Hippocampus

| | | |
|---|---|---|
| | | % Inhibition |
| Agent | Concentration | 0 ± SEM |

| | | | |
|---|---|---|---|
| Selegiline | 0.3 | μM | 28.3 ± 8.7 |
| | 5 | nM | 81.2 ± 2.6 |
| | 10 | nM | 95.6 ± 1.3 |
| | 30 | nM | 98.5 ± 0.5 |
| | 100 | nM | 98.8 ± 0.5 |
| | 30 | nM | 98.8 ± 0.5 |
| | 1 | μM | 99.1 ± 0.45 |
| R(−)DMS | 100 | nM | 59.4 ± 9.6 |
| | 300 | nM | 86.2 ± 4.7 |
| | 1 | μM | 98.2 ± 0.7 |
| | 3 | μM | 98.4 ± 0.95 |
| | 10 | μm | 99.1 ± 0.45 |
| | 30 | μM | 99.3 ± 0.40 |
| S(+)DMS | 300 | nM | 18.7 ± 2.1 |
| | 1 | μM | 44.4 ± 6.4 |
| | 3 | μM | 77.1 ± 6.0 |
| | 10 | μM | 94.2 ± 1.9 |
| | 30 | μM | 98.3 ± 0.6 |
| | 100 | μM | 99.3 ± 0.2 |
| | 1 | μm | 99.9 ± 0.1 |

TABLE 4
IC₅₀ Values for the Inhibition of MAO-B Guinea Pig

| | | Guinea Pig |
|---|---|---|
| Treatment | Human Platelets | Hippocampal Cortex |
| Selegiline | 5 nM (1) | 1 nM (1) |
| R(−)DMS | 400 nM (80) | 60 nM (60) |
| S(+)DMS | 1400 nM (2800) | 1200 nM (1200) |

( ) = reduction in potency compared to selegiline

As observed, R(−)DMS was 20-35 times more potent than S(+)DMS as an MAO-B inhibitor and both enantiomers were less potent than selegiline.

MAO-A Inhibitory Activity In Vitro

Results obtained from experiments examining the inhibition of MAO-A in guinea pig hippocampus are summarized in Table 5. The IC₅₀ values for the two enantiomers of DMS and for selegiline are shown in Table 6.

TABLE 5
MAO-A Inhibition in Guinea Pig Hippocampus

| | | |
|---|---|---|
| | | % Reduction |
| Agent | Concentration | 0 ± SEM |

| | | | |
|---|---|---|---|
| Selegiline | 300 | nM | 11.95 ± 2.4 |
| | 1 | μM | 22.1 ± 1.2 |
| | 3 | μM | 53.5 ± 2.7 |
| | 10 | μM | 91.2 ± 1.16 |
| | 100 | μM | 98.1 ± 1.4 |
| | 1 | mM | 99.8 ± 0.2 |
| R(−)DMS | 300 | nM | 4.8 ± 2.1 |
| | 1 | μM | 4.2 ± 1.5 |
| | 3 | μM | 10.5 ± 2.0 |
| | 10 | μM | 19.0 ± 1.3 |
| | 100 | μM | 64.2 ± 1.5 |
| | 1 | mM | 96.5 ± 1.2 |
| S(+)DMS | 1 | μM | 3.3 ± 1.5 |
| | 3 | μM | 4.3 ± 1.0 |
| | 10 | μM | 10.5 ± 1.47 |
| | 100 | μM | 48.4 ± 1.8 |
| | 1 | nM | 92.7 ± 2.5 |
| | 10 | nM | 99.6 ± 0.35 |

TABLE 6
IC₅₀ Values for the Inhibition of MAO-A

| | IC₅₀ for MAO-A in Guinea |
|---|---|
| Treatment | Pig Hippocampal Cortex |
| Selegiline | 2.5 μM (1) |
| R(−)DMS | 50.0 μM (20) |
| S(+)DMS | 100.0 μM (40) |

( ) = reduction in potency compared to selegiline

R(−)DMS was twice as potent as S(+)DMS as an MAO-A inhibitor and both were 20-40 times less potent than selegiline. Moreover, each of these agents were 2-3 orders of magnitude, i.e., 100 to 1000 times, less potent as inhibitors of MAO-A than inhibitors of MAO-B in hippocampal brain tissue. Therefore, selegiline and each enantiomer of DMS can be classified as selective MAO-B inhibitors in brain tissue.

Results of In Vivo Experiments

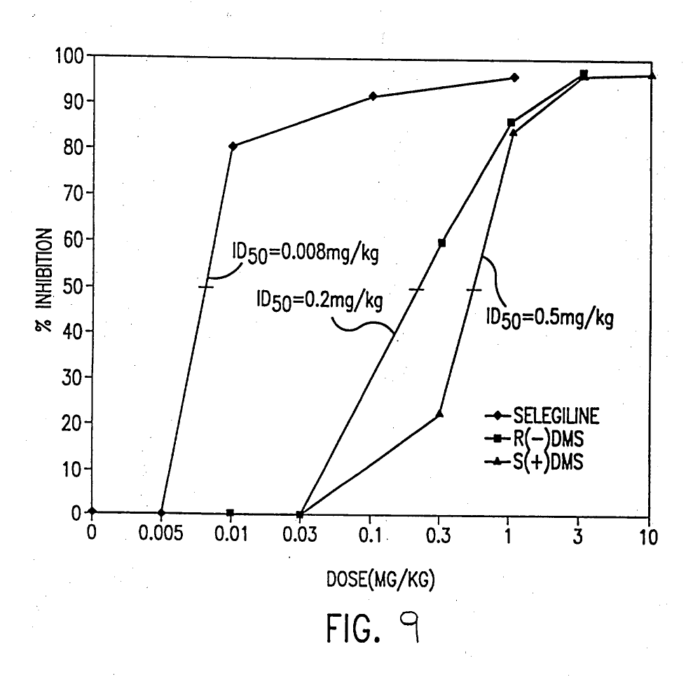

Each enantiomer of DMS was administered in vivo by subcutaneous injection once a day for five consecutive days, and inhibition of brain MAO-B activity was then determined. In preliminary studies, selegiline was found to have an ID₅₀ of 0.03 mg/kg; and both R(−)DMS and S(+)DMS were determined to be about 10 times less potent. More recent studies, performed on a larger group of animals, indicates that R(−)DMS is actually about 25 times less potent than selegiline as an inhibitor of MAO-B and that S(+)DMS is about 50 times less potent. Results are shown in FIG. 9 and ID₅₀ values are summarized in Table 7.

TABLE 7
ID₅₀ Values for Brain MAO-B Following
5 Days of Administration

| | ID₅₀ for MAO-B in Guinea |
|---|---|
| Treatment | Pig Hippocampal Cortex |
| Selegiline | 0.008 mg/kg (1) |
| R(−)DMS | 0.20 mg/kg (25) |
| S(+)DMS | 0.50 mg/kg (60) |

( ) - reduction in potency compared to selegiline

This experiment demonstrates that the enantiomers of DMS penetrate the blood brain-barrier and inhibit brain MAO-B after in vivo administration. It also demonstrates that the potency differences as an MAO-B inhibitor observed in vitro between each of the DMS enantiomers and selegiline are substantially reduced under in vivo conditions.

In experiments examining the effect of 5 s.c. treatments on MAO-A activity in guinea pig cortex (hippocampus), it was found that selegiline administration at a dose of 1.0 mg/kg resulted in a 36.1% inhibition of activity. R(−)DMS resulted in an inhibition of 29.8% when administered at a dose of 3.0 mg/kg. S(+)DMS administration did not cause any observable inhibition at the highest dose tested (10 mg/kg) indicating that it has significantly less cross reactivity potential.

C. Conclusions

In vitro, R(−)DMS and S(+)DMS both exhibit activity as MAO-B and MAO-A inhibitors. Each enantiomer was selective for MAO-B. S(+)DMS was less potent than R(−)DMS and both enantiomers of DMS were less potent than selegiline in inhibiting both MAO-A and MAO-B.

In vivo, both enantiomers demonstrated activity in inhibiting MAO-B, indicating that these enantiomers are able to cross the blood-brain barrier. The ability of these agents to inhibit MAO-B suggests that these agents may be of value as therapeutics for hypodopaminergic diseases such as ADHD and dementia.

### EXAMPLE 5

### In vivo Neuroprotection by the Enantiomers of Desmethylselegiline

The ability of the enantiomers of DMS to prevent neurological deterioration was examined by administering the agents to the wobbler mouse, an animal model of motor neuron disease, particularly amyotrophic lateral sclerosis (ALS). Wobbler mice exhibit progressively worsening forelimb weakness, gait disturbances, and flexion contractions of the forelimb muscles.

A. Test Method

A 0.1 mg/kg dose of R(−)DMS, S(+)DMS, or placebo was administered to wobbler mice by daily intra-peritoneal injection for a period of 30 days in a randomized, double-blind study. At the end of this time mice were examined for grip strength, running time, resting locomotive activity and graded for semi-quantitative paw posture abnormalities, and semi-quantitative walking abnormalities. The investigators who prepared and administered the test drugs to the animals were different than those who analyzed behavioral changes.

Assays and grading were performed essentially as described in Mitsumoto et al., Ann. Neurol. 36:142-148 (1994). Grip strength of the front paws of a mouse was determined by allowing the animal to grasp a wire with both paws. The wire was connected to a gram dynamometer and traction is applied to the tail of the mouse until the animal is forced to release the wire. The reading on the dynamometer at the point of release is taken as a measure of grip strength.

Running time is defined as the shortest time necessary to traverse a specified distance, e.g. 2.5 feet and the best time of several trials is recorded.

Paw posture abnormalities are graded on a scale based upon the degree of contraction and walking abnormalities are graded on a scale ranging from normal walking to an inability to support the body using the paws.

Locomotive activity is determined by transferring animals to an examination area in which the floor is covered with a square grid. Activity is measured by the number of squares traversed by a mouse in a set time interval, e.g., 9 minutes.

B. Results

At the beginning of the study, none of the groups were different in any variables, indicating that the three groups were comparative at the baseline. Weight gain was identical in all three groups, suggesting that no major side effects occurred in any animals. Table 8 summarizes differences that were observed in the mean grip strength of the test animals:

TABLE 8
Mean Grip Strength in Wobble Mice Treated
with R(−)DMS or S(+)DMS

| Treatment | N | Grip Strength (gm) |
|---|---|---|
| Control (placebo) | 10 | 9 (0-15) |
| R(−)DMS | 9 | 20 (0-63) |
| S(+)DMS | 9 | 14 (7-20) |

N = number of animals analyzed

Grip strength dropped markedly at the end of the first week in all animals. At the end of the study, grip strength was the least in control animals. The variability in grip strength in the treated animal groups prevented a meaningful statistical analysis of this data, however, at a dose of 0.1 mg/kg, the mean grip strength measured in the DMS-treated animals was greater than for the controls. These results suggest that the dose may have been too low, and that a higher dose study should be performed.

Running time, resting locomotive activity, semiquantitative paw posture abnormality grading, and semi-quantitative walking abnormality grading were also tested. None of these tests, however, showed any difference among the three groups tested.

### EXAMPLE 6

### Immune System Restoration by R(−)DMS and S(+)DMS

There is an age-related decline in immunological function that occurs in animals and humans which makes older individuals more susceptible to infectious disease and cancer. U.S. Pat. Nos. 5,276,057 and 5,387,615 suggest that selegiline is useful in the treatment of immune system dysfunction. The present experiments were undertaken to determine whether R(−)DMS and S(+)DMS are also useful in the treatment of such dysfunction. It should be recognized that an ability to bolster a patient's normal immunological defense's would be beneficial in the treatment of a wide variety of acute and chronic diseases including cancer, AIDS, both bacterial and viral infections, and some forms of peripheral neuropathy.

A. Test Procedure

The present experiments utilized a rat model to examine the ability of R(−)DMS and S(+)DMS to restore immunological function. Rats were divided into the following experimental groups:

1) young rats (3 months old, no treatment);

2) old rats (18-20 months old, no treatment);

3) old rats injected with saline;

4) old rats treated with selegiline at a dosage of 0.25 mg/kg body weight;

5) old rats treated with selegiline at a dosage of 1.0 mg/kg body weight;

6) old rats treated with R(−)DMS at a dosage of 0.025 mg/kg body weight;

7) old rats treated with R(−)DMS at a dosage of 0.25 mg/kg body. weight;

8) old rats treated with R(−)DMS at a dosage of 1.0 mg/kg body weight;

9) old rats treated with S(+)DMS at a dosage of 1.0 mg/kg body weight.

Rats were administered saline or test agent ip, daily for 60 days. They were then maintained for an additional “wash out” period of 10 days during which time no treatment was given. At the end of this time, animals were sacrificed and their spleens were removed. The spleen cells were then assayed for a variety of factors which are indicative of immune system function. Specifically, standard tests were employed to determine the following:

1) in vitro production of y-interferon by concanavalin A-stimulated spleen cells;

2) in vitro concanavalin A-induced production of interleukin-2;

3) percentage of IgM positive spleen cells (IgM is a marker of B lymphocytes);

4) percentage of CD5 positive spleen cells (CD5 is a marker of T lymphocytes).

B. Results

The effect of administration of selegiline, R(−)DMS and S(+)DMS on concanavalin A-induced interferon production by rat spleen cells is shown in Tables 9 and 10. Table 9 shows, that there is a sharp decline in cellular interferon production that occurs with age. Administration of selegiline, R(−)DMS, and S(+)DMS all led to a restoration of γ interferon levels with the most dramatic increases occurring at dosages of 1.0 mg/kg body weight.

TABLE 9
Effect of Age on T Cell Function*

| | |
|---|---|
| IL-2 | IFN-γ |

| Groups | U/ml | std. error | U/ml | std. error |
|---|---|---|---|---|
| young | 59.4 | 18.27 | 12297 | 6447 |
| old | 19.6 | 7.52 | 338 | 135 |

*T cell activities were assessed after stimulation of rat spleen cells with concanavalin A. TH, cytokines, IL-2 and IFN-γ were measured. Young vs. old, p = 0.0004

TABLE 10
Mean and % control IL-2 and IFN g

| | |
|---|---|
| IL-2 U/ml | IFN-γ U/ml |

| Groups | mean | % control | mean | % control |
|---|---|---|---|---|
| control* | 19.64 | 100 | 351 | 100 |
| control | 41.22 | 210 | 339 | 96 |
| R(−)DMS | 55.17 | 281 | 573 | 163 |
| R(−)DMS | 64.54 | 329 | 516 | 147 |
| R(−)DMS | 43.7 | 223 | 2728 | 777 |
| S(+)DMS | 57.12 | 291 | 918 | 261 |
| Sel 0.25 | 109.6 | 558 | 795 | 226 |
| Sel. 1.0 | 73.78 | 376 | 1934 | 550 |

*Old rats (22 months old) with no treatment

Table 10 shows the extent to which R(−)DMS, S(+)DMS and selegiline are capable of restoring y-interferon production in the spleen cells of old rats. Interferon-γ is a cytokine associated with T cells that inhibit viral replication and regulate a variety of immunological functions. It influences the class of antibodies produced by B-cells, upregulates class I and class II MHC complex antigens and increases the efficiency of macrophage-mediated killing of intracellular parasites.

Histological immunofluorescence studies show a dramatic loss of innervation in rat spleens with age. When rats are treated with R(−)DMS, there is a significant increase in innervation in the spleens of animals and this increase occurs in a dose-response manner. S(+)DMS did not show any effect on histological examination, despite a modest increase in interferon-γ production. IL-2 production was not enhanced by treatment with R(−)DMS or S(+)DMS, suggesting that the effects of these agents may be limited to IFN-γ production.

C. Conclusions

The results obtained with respect to histological examination, the production of interferon, and the percentage of IgM positive spleen cells support the conclusion that the DMS enantiomers are capable of at least partially restoring the age-dependent loss of immune system function. The results observed with respect to IFN-y are particularly important. In both humans and animals, IFN-y production is associated with the ability to successfully recover from infection with viruses and other pathogens. In addition, it appears that R(−)DMS and S(+)DMS will have a therapeutically beneficial effect for diseases and conditions mediated by weakened host immunity. This would include AIDS, response to vaccines, infectious diseases, adverse immunological effects caused by cancer chemotherapy and cancer, and some forms of peripheral neuropathy.

### EXAMPLE 7

### Examples of Dosage Forms

A. Desmethylselegiline Patch

| Dry Weight Basis | |
|---|---|
| Component | (mg/cm²) |
| Durotak ® 87-2194 | 90 parts by weight |
| adhesive acrylic polymer | |
| Desmethylselegiline | 10 parts by weight |

The two ingredients are thoroughly mixed, cast on a film backing sheet (e.g., Scotchpak® 9723 polyester) and dried. The backing sheet is cut into patches a fluoropolymer release liner (e.g., Scotchpak® 1022) is applied, and the patch is hermetically sealed in a foil pouch. One patch is applied daily to supply 1-10 mg of desmethylselegiline per 24 hours in the treatment of conditions in a human produced by neuronal degeneration or neuronal trauma.

B. Ophthalmic Solution

Desmethylselegiline (0.1 g) as the hydrochloride, 1.9 g of boric acid, and 0.004 g of phenyl mercuric nitrate are dissolved in sterile water qs 100 ml. The mixture is sterilized and sealed. It can be used ophthalmologically in the treatment of conditions produced by neuronal degeneration or neuronal trauma, as for example glaucomatous optic neuropathy and macular degeneration.

C. Intravenous Solution

A 1% solution is prepared by dissolving 1 g of desmethylselegiline as the HCl in sufficient 0.9% isotonic saline solution to provide a final volume of 100 ml. The solution is buffered to pH 4 with citric acid, sealed, and sterilized to provide a 1% solution suitable for intravenous administration in the treatment of conditions produced by neuronal degeneration or neuronal trauma.

D. Oral Dosage Form

Tablets and capsules containing desmethylselegiline are prepared from the following ingredients (mg/unit dose):

| | |
|---|---|
| desmethylselegiline | 1-5 |
| microcrystalline cellulose | 86 |
| lactose | 41.6 |
| citric acid | 0.5-2 |
| sodium citrate | 0.1-2 |
| magnesium Stearate | 0.4 |

with an approximately 1:1 ratio of citric acid and sodium citrate.

### EXAMPLE 8

### Treatment of a Mouse Model of Cisplatin-Induced Neuropathy by R(−)DMS

The ability of desmethylselegiline to treat peripheral neuropathy in a mouse model of cisplatin-induced neuropathy was investigated. Male CD1 mice weighing between 15 and 20 grams at the outset of the experiment were divided into six groups of 15 and dosed as follows:

| | |
|---|---|
| Group 1: | control-saline plus buffer only. |
| Group 2: | cisplatin plus buffer. |
| Group 3: | cisplatin plus selegiline. |
| Group 4: | selegiline only. |
| Group 5: | cisplatin plus R(−)-desmethylselegiline. |
| Group 6: | R(−)-desmethylselegiline alone. |

The cisplatin was administered to the mice by intraperitoneal injection at a dose of 10 mg/kg body weight once a week for eight (8) consecutive weeks. Selegiline and R(−)-desmethylselegiline were administered subcutaneously to the mice at a dose of 1 mg/kg body weight five (5) times a week for eight consecutive weeks. Additionally, the mice were given a daily subcutaneous injection of saline to maintain hydration and normal kidney function.

After 8 full weeks of cisplatin therapy, the following number of mice as shown in Table 11 survived in each group from an initial count of 15:

TABLE 11
Survival of Treated Mice

| | | |
|---|---|---|
| Group 1: | 14 | (control) |
| Group 2: | 12 | (cisplatin) |
| Group 3: | 11 | (cisplatin + selegiline) |
| Group 4: | 15 | (selegiline) |
| Group 5: | 7 | (cisplatin + R(−)-desmethylselegiline) |
| Group 6: | 13 | (R(−)-desmethylselegiline) |

With the exception of the group receiving cisplatin and R(−)-desmethylselegiline, there were fewer deaths than typically encountered in studies of cisplatin peripheral neuropathy. This may be due to the aggressive hydration with saline injection each day during the experiment.

All behavioral testing of the surviving mice described in this Example was performed on the day following the last dose of selegiline and R(−)-desmethylselegiline to the mice. Cisplatin characteristically produces a large fiber sensory neuropathy. The tailflick test was used to examine the function of small fiber sensory neurons in the groups of mice. This test measures an animal's response to a thermal noxious stimulus via a spinal cord mediated reflex. The tailflick test was performed by loosely restraining the mice and exposing their tails to a focused light beam at a set distance. The latency period for the mice to withdraw their tails from the beam was then measured. While a significant alteration in the tailflick threshold has been observed with severe cisplatin-induced neuropathies, this has been a variable finding because the small fiber neurons are not the primary population sensitive to cisplatin. As shown below in Table 12, no significant difference were found between the surviving members of the different groups with respect to tailflick threshold:

TABLE 12
Tailflick Threshold

| | |
|---|---|
| Control: | 7.0 ± 0.3 seconds (mean ± SEM) |
| Cisplatin: | 7.8 ± 0.8 seconds (mean ± SEM) |
| Cisplatin + Selegiline: | 7.9 ± 0.5 seconds (mean ± SEM) |
| Selegiline: | 8.7 ± 0.6 seconds (mean ± SEM) |
| Cisplatin + R(−)- | 7.4 ± 0.8 seconds (mean ± SEM) |
| desmethylselegiline: | |
| R(−)-desmethylselegiline: | 6.9 ± 0.4 seconds (mean ± SEM) |

Proprioceptive testing was used to assess the effect of selegiline and R(−)-desmethylselegiline on peripheral nerve function in mice with cisplatin-induced neuropathy. Proprioception is a large fiber sensory modality that is typically abnormal in the presence of cisplatin-induced peripheral neuropathy. Proprioceptive testing analyzes the function of large fiber sensory neurons by measuring the ability of mice to maintain their balance on a rotating dowel with visual cues removed. This ability requires the mouse to feel where its limbs are in space, as well as where the dowel is rotating, which are proprioceptive functions.

The mice were placed on a rotating dowel in a completely dark room and timed until they fell off the dowel, for a maximum of 20 seconds. The results of this test shown in Table 13 were highly significant and suggest that selegiline and R(−)-desmethylselegiline beneficially protects mice against cisplatin-induced peripheral neuropathy:

TABLE 13
Proprioceptive Test

| | |
|---|---|
| Control: | 18 ± 1.3* seconds (mean ± SEM) |
| Cisplatin: | 8.3 ± 2.6 seconds (mean ± SEM) |
| Cisplatin + Selegiline: | 14.8 ± 1.7* seconds (mean ± SEM) |
| Selegiline: | 16.4 ± 1.7* seconds (mean ± SEM) |
| Cisplatin + R(−)- | 20 ± 0* seconds (mean ± SEM) |
| desmethylselegiline: | |
| R(−)-desmethylselegiline: | 17.1 ± 1.1* seconds (mean ± SEM) |

The overall p value was 0.0004 by ANOVA. The approximate p value using the Krukal-Wallis nonparametric AVOVA test was 0.0035. Individual comparisons were made using Student-Newman-Keuls multiple comparisons test. Indicates that this group differed from the cisplatin group with a p<0.05.

As seen in the above data, apart from the cisplatin-treated group, none of the other groups differed significantly from the control group. Additionally, the mice in the cisplatin plus R(−)-desmethylselegiline group were the most successful group of mice in the proprioceptive test, because unlike the cisplatin plus selegiline group, all the mice in this group were able to stay on the dowel for the entire 20 second time period, despite being treated with cisplatin.

Since cisplatin primarily effects large fiber sensory function, it will typically cause abnormalities of nerve conduction velocity in sensory nerves. The large, well myelinated fibers make the major contribution to measured conduction velocity; therefore, this measure may be impaired in mice with cisplatin-induced neuropathy. Action potential amplitudes are primarily determined by axonal integrity so it is less likely to be affected. All groups of mice underwent electrophysiological testing one week following their last dose of selegiline or R(−)-desmethylselegiline. Measurements were taken of the conduction velocity and action potential amplitudes of the compound caudal nerve which runs through the tail. As shown below in Table 14, the data suggests that cisplatin significantly reduces the nerve conduction velocity, and that this effect was not prevented by either selegiline or R(−)-desmethylselegiline administration. There were no statistically significant differences between the groups treated with cisplatin with respect to the action potential amplitudes:

TABLE 14
Electrophysiological Studies

| | | Distance | Temp | Latency | Amplitude | NCV |
|---|---|---|---|---|---|---|
| Control | Mean | 40 mm | 35.7 | 1.25 | 62.8 | 32.3 |
| | SD | | 0.7 | 0.15 | 13.6 | 3.2 |
| Cisplatin | Mean | 40 mm | 34.2 | 1.45 | 59.7 | 27.8* |
| | SD | | 1.2 | 0.14 | 17.2 | 2.6 |
| Cisplatin + | Mean | 40 mm | 33.7 | 1.43 | 81.83 | 28.5* |
| Selegiline | SD | | 0.5 | 0.13 | 19.4 | 2.5 |
| Selegiline | Mean | 40 mm | 35.4 | 1.25 | 52.65 | 32.3 |
| | SD | | 1.2 | 0.12 | 16.8 | 2.8 |
| Cisplatin+ | Mean | 40 mm | 34.1 | 1.6 | 45.28 | 25.6* |
| R(−)DMS | SD | | 1.8 | 0.11 | 5.9 | 1.8 |
| R(−)DMS | Mean | 40 mm | 35.8 | 1.3 | 56.0 | 31.2 |
| | SD | | 0.8 | 0.1 | 8.2 | 3.3 |

The overall p value was 0.0001 by ANOVA for conduction velocity. Comparisons between groups were performed using Student-Newman-Keuls multiple comparisons test. * Indicates that this group differed from the control group with a p<0.05.

After the electrophysiological testing, the mice were sacrificed and the four dorsal root ganglia were removed and assayed for the neuropeptide calcitonin gene related peptide (CGRP), using radioimmunoassay. CGRP is a ubiquitous neuropeptide that is primarily associated with small fiber sensory neurons, but it is also expressed in large fiber neurons. CGRP is thought to play a role in mediating pain sensation, but it may also have a broader role in the dorsal root ganglion. The level of CGRP was assayed because it has been found that CGRP is significantly reduced in dorsal root ganglia following exposure to cisplatin. As expected, a significant reduction in CGRP expression was found in mice treated with cisplatin. This reduction in CGRP expression was not ameliorated in mice also treated with selegiline or R(−)-desmethylselegiline, as shown in Table 15.

TABLE 15
CGRP Levels

| | |
|---|---|
| Control: | 424.8 ± 27 fmol/ganglion (mean ± SEM) |
| Cisplatin: | 163.2 ± 30.6* fmol/ganglion (mean ± SEM) |
| Cisplatin + Selegiline: | 238.2 ± 27.6* fmol/ganglion (mean ± SEM) |
| Selegiline: | 372.9 ± 33.3 fmol/ganglion (mean ± SEM) |
| Cisplatin + R(−)- | 227.4 ± 51.6* fmol/ganglion (mean ± SEM) |
| desmethylselegiline: | |
| R(−)-desmethylselegiline: | 331.8 ± 18.3 fmol/ganglion (mean ± SEM) |

The overall p value was 0.0001 by ANOVA. Individual comparisons were made using Student-Newman-Keuls multiple comparisons test. * Indicates that this group differed from the control group with a p<0.05.

As shown by the above date, cisplatin was able to induce sensory peripheral neuropathy in surviving mice. Cisplatin-treated mice demonstrated significant differences from control mice in proprioception, nerve conduction velocity, and sensory ganglion expression of CGRP. Animal that were also treated with selegiline or R(−)-desmethylselegiline did markedly better than mice treated with cisplatin alone in the behavioral measure of proprioceptive function. Neither selegiline or R(−)-desmethylselegiline, however, appear to prevent the changes in nerve conduction velocity and CGRP expression resulting from treatment with cisplatin. One possible explanation is that functional proprioception is dependent on factors other than those aspects of normal neuronal function that are responsible for nerve conduction velocity and CGRP expression. Since CGRP is not known to be specifically expressed in the large fiber neurons responsible for proprioceptive sensation, it is not surprising that there would be such a dichotomy. Also, the functional significance of CGRP expression, and its relevance to clinical neuropathy, is unclear.

### EXAMPLE 9

### Treatment of Peripheral Neuropathy Caused by Vincristine

A patient with endometrial carcinoma is given an intravenous bolus injection of vincristine at a dose of 1.4 mg/m² weekly. The toxic effects of vincristine cause sensory loss in the fingers and toes, a loss of the ankle jerk reflex, weakness, and postural hypotension. The patient is administered 5 mg of R(−)DMS and/or S(+)DMS orally twice a day, once with breakfast and once at lunch. During this time, therapy with vincristine is continued and evaluations of both tumor response and toxic side effects are carried out by a physician on a weekly basis. After continued therapy, symptoms associated with peripheral neuropathy subside. At this point, the dosage of vincristine is increased to 1.8 mg/m² and the process is continued. If symptoms of peripheral neuropathy do not return at the end of another cycle of chemotherapy, dosage is increased again until an upper limit is reached. After the final dose of vincristin is given, R(−)DMS and/or S(+)DMS administration is maintained for a period of one month.

### EXAMPLE 10

### Administration of Desmethylselegiline Enantiomers in Combination With Cisplatin

A patient with ovarian cancer is given weekly injections of cisplatin at a dosage of 120 mg/m². Concurrently, the patient is given an oral dose of 5 mg of R(−)DMS and/or S(+)DMS twice a day. At the end of one week, the patient is evaluated for signs of peripheral neuropathy. If no symptoms appear, the dose of R(−)DMS and/or S(+)DMS is maintained and the dosage of cisplatin is increased to 140 mg/m² per week. This process is continued until an upper limit of cisplatin is identified. The effect of the therapy on tumor progression is evaluated to determine the efficacy of the treatment.

### EXAMPLE 11

### Treatment of Peripheral Neuropathy Caused by Paclitaxel

A patient with breast cancer is administered R(−)DMS and/or S(+)DMS orally (10 mg per day) for a period of one week. At the end of this time, treatment with paclitaxel is begun by infusing the drug intravenously at a dose of 175 mg/m² over a period of 3 hours. Treatment is repeated every 3 weeks for a total of ten cycles, with the dosage of paclitaxel being increased by 25 mg/m² at each cycle. During this time, treatment with R(−)DMS and/or S(+)DMS is continued and evaluations of both tumor response and toxic side effects are carried out by a physician on a weekly basis. Dosage of paclitaxel continues to be increased until side effects become unacceptably severe. Administration of R(−)DMS and/or S(+)DMS is continued for one month after treatment with paclitaxel ends.

### EXAMPLE 12

### Alternative Therapeutic Regime Using Paclitaxel and R(−)DMS and/or S(+)DMS

A patient with breast cancer is administered R(−)DMS and/or S(+)DMS via a transdermal patch at a dose of about 0.10 mg/kg per day for a period of one week. At the end of this time, treatment with paclitaxel is begun by infusing the drug intravenously at a dose of 175 mg/m² over a period of 3 hours. Paclitaxel infusion is repeated every 3 weeks. During this time, treatment with R(−)DMS and/or S(+)DMS is continued and evaluations of both tumor response and toxic side effects are carried out by a physician on a weekly basis. If peripheral neuropathy becomes unacceptably severe the dosage of R(−)DMS and/or S(+)DMS is increased to about 0.15 mg/kg per day. If unacceptable side effects persist, the dosage of paclitaxel is reduced to 125 mg/m². Treatment cycles are continued for a period extending as long as a beneficial effect on tumor progression is obtained or until unacceptable side effects can no longer be eliminated. Administration of R(−)DMS and/or S(+)DMS is continued for one month after treatment with paclitaxel ends.

### EXAMPLE 13

### Treatment of Peripheral Neuropathy Caused by Diabetic Neuropathy

R(−)DMS and/or S(+)DMS is administered orally (10 mg per day) to a patient with diabetes who is not yet suffering from diabetic neuropathy. This early treatment with R(−)DMS and/or S(+)DMS is periodically evaluated by a physician to determine whether the patient develops any diabetic neuropathies. Long-term administration of R(−)DMS and/or S(+)DMS is continued to reduce the likelihood of or eliminate the development of diabetic neuropathy in the patient. In a patient with diabetes who presents with a diabetic neuropathy, R(−)DMS and/or S(+)DMS is administered orally (20 mg per day) to reduce and/or reverse the symptoms of the diabetic neuropathy. Treatment is continued until the symptoms are reduced or eliminated, and then 10 mg of R(−)DMS and/or S(+)DMS is administered orally to the patient per day to reduce the likelihood of or eliminate the development of subsequent diabetic neuropathies.

### EXAMPLE 14

### Treatment of Peripheral Neuropathy Caused by Alcoholic Neuropathy

A patient suffering from alcoholic peripheral neuropathy is administered R(−)DMS and/or S(+)DMS via a transdermal patch at a dose of about 0.05 mg/kg per day. This treatment with R(−)DMS and/or S(+)DMS is periodically evaluated by a physician to determine whether the patient continues to suffer from alcoholic neuropathy. Long-term administration of R(−)DMS and/or S(+)DMS may be necessary until the cause of the alcoholic neuropathy is eliminated by the patient.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of preventing or treating peripheral neuropathy caused by a toxic agent in a subject in need of such prevention or treatment, comprising:
administering R(−)-desmethylselegiline to the subject in an amount sufficient to prevent, reduce, or eliminate one or more of the symptoms associated with the peripheral neuropathy.

2. The method of claim 1, wherein the toxic agent that causes peripheral neuropathy is selected from the group consisting of a drug, an industrial chemical, and an environmental toxin.

3. The method of claim 2, wherein the drug is chloramphenicol, colchicine, dapsone, disulfiram, amiodarone, gold, isoniazid, misonidazole, nitrofurantoin, perhexiline, propafenone, pyridoxine, phenytoin, simvastatin, tacrolimus, thalidomide, or zalcitabine.

4. The method of claim 1, wherein the toxic agent is acrylamide, arsenic, carbon disulfide, hexacarbons, lead, mercury, platinum, an organophosphate, or thallium.

5. The method of claim 1, wherein the toxic agent is a chemotherapeutic agent.

6. The method of claim 5, wherein the chemotherapeutic agent is administered for the treatment of cancer.

7. The method of claim 5, wherein the chemotherapeutic agent is selected from the group consisting of cisplatin, paclitaxel, vincristine, and vinblastin.

8. The method of claim 1, wherein the toxic agent is alcohol.

9. The method of claim 1, wherein the R(−)-desmethylselegiline is administered by a route that avoids absorption of R(−)-desmethylselegiline from the gastrointestinal tract.

10. The method of claim 9, wherein the R(−)-desmethylselegiline is administered transdermcanally, buccally, sublingually, or parenterally.

11. The method of claim 1, wherein the patient is a human.

12. The method of claim 1, wherein the R(−)-desmethylselegiline is administered at a dose of between 0.01 mg/kg per day and 0.15 mg/kg per day based upon the weight of the free amine.

13. A method of treating a subject for peripheral neuropathy caused by a genetically inherited condition, comprising:
administering R(−)-desmethylselegiline to the subject in an amount sufficient to reduce or eliminate one or more of the symptoms associated with the peripheral neuropathy.

14. The method of claim 13, wherein the genetically inherited condition that causes peripheral neuropathy is selected from the group consisting of Charcot-Marie-Tooth Disease, Dejerine-Sottas Disease, Riley-Day Syndrome, Porphyrias, Giant Axonal Neuropathy, and Friedrich's ataxia.

15. The method of claim 13, wherein the patient is a human.

16. The method of claim 13, wherein the R(−)-desmethylselegiline is administered by a route that avoids absorption of R(−)-desmethylselegiline from the gastrointestinal tract.

17. The method of claim 16, wherein the R(−)-desmethylselegiline is administered transdermally, buccally, sublingually, or parenterally.

18. The method of claim 13, wherein the R(−)-desmethylselegiline is administered at a dose of between 0.01 mg/kg per day and 0.15 mg/kg per day based upon the weight of the free amine.

19. A method of preventing or treating a subject for peripheral neuropathy caused by a systemic disease, comprising:
administering R(−)-desmethylselegiline to the subject in an amount sufficient to reduce or eliminate one or more of the symptoms associated with the peripheral neuropathy.

20. The method of claim 19, wherein the peripheral neuropathy is selected from the group consisting of acquired primary demyelinating neuropathy, distal symmetric sensory polyneuropathy, distal symmetric sensorimotor polyneuropathy, vasculitic neuropathy, infectious neuropathy, idiopathic neuropathy; immune-mediated neuropathy; nutrition-related neuropathy, and paraneoplastic neuropathy.

21. The method of claim 20, wherein the acquired primary demyelinating neuropathy is chronic inflammatory demyelinating polyradiculoneuropathy (CIDP), acute inflammatory demyelinating polyneuropathy (AIDP), or Guillain-Barre syndrome.

22. The method of claim 20, wherein the infectious neuropathy is caused by herpes simplex, herpes zoster, hepatitis B, hepatitis C, HIV, cytomegalovirus, diphtheria, leprosy, or Lyme disease.

23. The method of claim 19, wherein the systemic disease is alcoholic polyneuropathy.

24. The method of claim 19, wherein the systemic disease is diabetes mellitus.

25. The method of claim 19, wherein the systemic disease is pernicious anemia.

26. The method of claim 19, wherein the systemic disease is uremia, rheumatoid arthritis, sarcoidosis, or hypothyroidism.

27. The method of claim 19, wherein the patient is a human.

28. The method of claim 19, wherein the R(−)-desmethylselegiline is administered by a route that avoids absorption of R(−)-desmethylselegiline from the gastrointestinal tract.

29. The method of claim 28, wherein the R(−)-desmethylselegiline is administered transdermally, buccally, sublingually, or parenterally.

30. The method of claim 19, wherein the R(−)-desmethylselegiline is administered at a dose of between 0.01 mg/kg per day and 0.15 mg/kg per day based upon the weight of the free amine.

31. A method for treating a subject with cancer comprising:
a) administering to the subject a chemotherapeutic agent known to have a toxic effect on peripheral nerves, wherein the chemotherapeutic agent is administered at a dose effective at slowing the progression of the cancer; and
b) concurrently administering R(−)-desmethylselegiline to the subject at a dose effective at reducing or eliminating the peripheral neuropathy associated with the chemotherapeutic agent.

32. The method of claim 31, wherein the subject is a human.

33. The method of claim 31, wherein the chemotherapeutic agent is cisplatin, paclitaxel, vincristine, or vinblastin.

34. The method of claim 31, wherein the R(−)-desmethylselegiline is administered by a route that avoids absorption of R(−)-desmethylselegiline from the gastrointestinal tract.

35. The method of claim 34, wherein the R(−)-desmethylselegiline is administered transdermally, buccally, sublingually, or parenterally.

36. The method of claim 31, wherein the R(−)-desmethylselegiline is administered at a daily dose of between 0.01 mg/kg and about 0.15 mg/kg, calculated on the basis of the free secondary amine.

37. A method of preventing or treating a subject for peripheral neuropathy caused by compression, trauma, or entrapment, comprising:
administering R(−)-desmethylsclcgiline to the subject in an amount sufficient to reduce or eliminate one or more of the symptoms associated with the peripheral neuropathy.

38. The method of claim 37, wherein the peripheral neuropathy is a compression neuropathy selected from the group consisting of carpal tunnel syndrome, ulnar neuropathy at the elbow or wrist, common peroneal nerve at the knee, tibial nerve at the knee, and sciatic nerve.

39. The method of claim 37, wherein the patient is a human.

40. The method of claim 37, wherein the R(−)-desmethylselegiline is administered by a route that avoids absorption of R(−)-desmethylselegiline from the gastrointestinal tract.

41. The method of claim 40, wherein the R(−)-desmethylselegiline is administered transdermally, buccally, sublingually, or parenterally.

42. The method of claim 37, wherein the R(−)-desmethylselegiline is administered at a dose of between 0.01 mg/kg per day and 0.15 mg/kg per day based upon the weight of the free amine.

43. A method of preventing or treating large-fiber peripheral neuropathy in a subject in need of such prevention or treatment, comprising:
administering R(−)-desmethylselegiline to the subject in an amount sufficient to prevent, reduce, or eliminate one or more of the symptoms associated with the large-fiber peripheral neuropathy.

44. The method of claim 43, wherein the large-fiber peripheral neuropathy is a large-fiber sensory neuropathy.

45. The method of claim 43, wherein the large-fiber peripheral neuropathy is a large-fiber motor neuropathy.

46. A method of preventing or treating small-fiber peripheral neuropathy in a subject in need of such prevention or treatment, comprising:
administering R(−)-desmethylselegiline to the subject in an amount sufficient to prevent, reduce, or eliminate one or more of the symptoms associated with the small-fiber peripheral neuropathy.

47. The method of claim 46, wherein the small-fiber peripheral neuropathy results from abnormal function or pathological change in small, myelinated axons.

48. The method of claim 46, wherein the small-fiber peripheral neuropathy results from abnormal function or pathological change in small, unmyelinated axons.

49. A method of preventing or treating a subject for autonomic peripheral neuropathy in a subject in need of such prevention or treatment, comprising:
administering R(−)-desmethylselegiline to the subject in an amount sufficient to reduce or eliminate one or more of the symptoms associated with the autonomic peripheral neuropathy.

50. The method of claim 49, wherein the autonomic peripheral neuropathy results from the dysfunction of peripheral autonomic nerves.

51. The method of claim 50, wherein the peripheral autonomic nerves are small, myelinated nerves.

52. A method of preventing or treating a motor neuron disease in a subject in need of such prevention or treatment, comprising:
administering R(−)-desmethylselegiline to the subject in an amount sufficient to reduce or eliminate one or more of the symptoms associated with the motor neuron disease.

53. The method of claim 52, wherein the motor neuron disease results from the degeneration of upper motor neurons, lower motor neurons, or upper and lower motor neurons.

54. The method of claim 53, wherein the motor neuron disease results from the degeneration of lower motor neurons.

55. The method of claim 52, wherein the motor neuron disease is selected from the group consisting of Progressive Bulbar Palsy, Spinal Muscular Atrophy, Kugelberg-Welander Syndrome, Duchenne's Paralysis, Postpolio Syndrome, Werdnig-Hoffman Disease, Kennedy's Disease, and Benign Focal Amyotrophy.

56. The method of claim 52, wherein the motor neuron disease is amyotrophic lateral sclerosis.

57. A pharmaceutical composition, comprising:
a) R(−)-desmethylselgiline; and
b) a second therapeutic agent useful in the treatment of peripheral neuropathy.

58. The composition of claim 57, wherein the second therapeutic agent is selected from the group consisting of prednisone, IVIg, cyclophosphamide, famciclovir, tegretol, tricyclic antidepressants, dapsone, clofazamine, rifampin, nifurtimox, benznidaxole, gabapentin, ganciclovir, foscarnet, cidofovir, acyclovir, topical Lidocaine, and ribavirin.

59. The composition of claim 57, wherein between about 0.015 and about 5.0 mg/kg of R(−)-desmethylselgiline, calculated on the basis of the free secondary amine, is in a unit dose of the composition.

60. The composition of claim 57, for oral administration.

61. The composition of claim 57, for non-oral administration.

62. The composition of claim 57, wherein the composition is a transdermal patch.
